(19) United States
(12) Patent Application Publication
(10) Pub. No.: US 2015/0119378 A1
(43) Pub. Date: Apr. 30, 2015

(54) AZETIDINYLOXY-, PYRROLIDINYLOXY-, AND PIPERIDINYLOXY-SUBSTITUTED METANICOTINES AS NEURONAL NICOTINIC ACETYLCHOLINE RECEPTOR LIGANDS

(71) Applicant: Targacept, Inc., Winston-Salem, NC (US)

(72) Inventors: Srinivasa Rao Akireddy, Winston-Salem, NC (US); Scott R. Breining, Winston-Salem, NC (US)

(21) Appl. No.: 14/522,767

(22) Filed: Oct. 24, 2014

Related U.S. Application Data
(60) Provisional application No. 61/895,430, filed on Oct. 25, 2013.

(51) Int. Cl. C07D 405/14; C07D 401/12

(52) U.S. Cl. CPC C07D 405/14; C07D 401/12
USPC 514/210.18; 546/278.4; 514/343; 546/282.1

(57) ABSTRACT

The present invention relates to compounds that bind to and modulate the activity of neuronal nicotinic acetylcholine receptors, to processes for preparing these compounds, to pharmaceutical compositions containing these compounds, and to methods of using these compounds for treating a wide variety of conditions and disorders, including those associated with dysfunction of the central nervous system (CNS).

### CROSS-REFERENCE TO RELATED APPLICATION(S)

The present application is a non-provisional claiming priority to U.S. Provisional Application Ser. No. 61/895,430, filed Oct. 25, 2013, herein incorporated by reference in its entirety.

### FIELD OF THE INVENTION

The present invention relates to compounds that bind to and modulate the activity of neuronal nicotinic acetylcholine receptors, to processes for preparing these compounds, to pharmaceutical compositions containing these compounds, and to methods of using these compounds for treating a wide variety of conditions and disorders, including those associated with dysfunction of the central nervous system (CNS).

### BACKGROUND OF THE INVENTION

The therapeutic potential of compounds that target neuronal nicotinic receptors (NNRs), also known as nicotinic acetylcholine receptors (nAChRs), has been the subject of several reviews. See, for example, Arneric et al., Biochem. Pharmacol. 74: 1092 (2007), Breining et al., Ann. Rep. Med. Chem. 40: 3 (2005), Hogg and Bertrand, Curr. Drug Targets: CNS Neurol. Disord. 3: 123 (2004), Suto and Zacharias, Expert Opin. Ther. Targets 8: 61 (2004), Dani et al., Bioorg. Med. Chem. Lett. 14: 1837 (2004), Bencherif and Schmitt, Curr. Drug Targets: CNS Neurol. Disord. 1: 349 (2002). Among the kinds of indications for which NNR ligands have been proposed as therapies are cognitive disorders, including Alzheimer's disease, attention deficit disorder, and schizophrenia (Biton et al., Neuropsychopharm. 32: 1 (2007), Boess et al., J. Pharmacol. Exp. Ther. 321: 716 (2007), Hajos et al., J. Pharmacol. Exp. Ther. 312: 1213 (2005), Newhouse et al., Curr. Opin. Pharmacol. 4: 36 (2004), Levin and Rezvani, Curr. Drug Targets: CNS Neurol. Disord. 1:423 (2002), Graham et al., Curr. Drug Targets: CNS Neurol. Disord. 1: 387 (2002), Ripoll et al., Curr. Med. Res. Opin. 20(7): 1057 (2004), and McEvoy and Allen, Cuff. Drug Targets: CNS Neurol. Disord. 1: 433 (2002)); pain and inflammation (Decker et al., Curr. Top. Med. Chem. 4(3): 369 (2004), Vincler, Expert Opin. Invest. Drugs 14(10): 1191 (2005), Jain, Curr. Opin. Inv. Drugs 5: 76 (2004), Miao et al., Neuroscience 123: 777 (2004)); depression and anxiety (Shytle et al., Mol. Psychiatry 7: 525 (2002), Damaj et al., Mol. Pharmacol. 66: 675 (2004), Shytle et al., Depress. Anxiety 16: 89 (2002)); neurodegeneration (O'Neill et al., Curr. Drug Targets: CNS Neurol. Disord. 1: 399 (2002), Takata et al., J. Pharmacol. Exp. Ther. 306: 772 (2003), Marrero et al., J. Pharmacol. Exp. Ther. 309: 16 (2004)); Parkinson's disease (Bordia et al., J Pharmacol. Exp. Ther. 327: 239 (2008), Jonnala and Buccafusco, J. Neurosci. Res. 66: 565 (2001)); addiction (Dwoskin and Crooks, Biochem. Pharmacol. 63: 89 (2002), Coe et al., Bioorg. Med. Chem. Lett. 15(22): 4889 (2005)); obesity (Li et al., Curr. Top. Med. Chem. 3: 899 (2003)); and Tourette's syndrome (Sacco et al., J. Psychopharmacol. 18(4): 457 (2004), Young et al., Clin. Ther. 23(4): 532 (2001))

There exists a heterogeneous distribution of nAChR subtypes in both the central and peripheral nervous systems. For instance, the α4β2, α6β2*, α7, and α3β2 subtypes are predominant in vertebrate brain, whereas the α3β4 subtype is predominate at the autonomic ganglia, and the α1β1δγ and α1β1δε subtypes are predominant at the neuromuscular junction (see Dwoskin et al., Exp. Opin. Ther. Patents 10: 1561 (2000) and Holliday et al. J. Med. Chem. 40(26), 4169 (1997)). Compounds which selectively target the CNS predominant subtypes have potential utility in treating various CNS disorders. However, a limitation of some nicotinic compounds is that they lack the selectivity required to preferentially target CNS receptors over receptor located in the muscle and ganglion. Such drugs are often associated with various undesirable side effects. Therefore, there is a need to have compounds, compositions, and methods for preventing or treating various conditions or disorders where the compounds exhibit a high enough degree of nAChR subtype specificity to elicit a beneficial effect, without significantly affecting those receptor subtypes which have the potential to induce undesirable side effects, including, for example, appreciable activity at cardiovascular and skeletal muscle sites.

### SUMMARY OF THE INVENTION

The present invention includes compounds of Formula I:

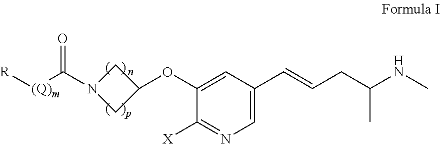

wherein:

n is 0, 1, 2, 3, or 4;

p is 0, 1, 2, 3, or 4;

and the sum of n and p is 2, 3, or 4;

m is 0 or 1;

Q is —NH— or —O—;

X is H or halogen;

R is chosen from the group consisting of
C₁₋₆ alkyl, optionally substituted with one or more of fluorine or
C₁₋₆ alkoxy,
cycloalkyl, optionally substituted with one or more fluorine atoms,
C₃₋₆ heterocyclyl, optionally substituted with one or more of fluorine, C₁₋₆ alkyloxy, and C₁₋₆ alkyl optionally substituted with one or more fluorine atoms,
heteroaryl, optionally substituted with one or more fluorine atoms, and
aryl, optionally substituted with one or more fluorine atoms;

or a pharmaceutically acceptable salt thereof.

The compounds of the present invention bind with high affinity to NNRs of the α4β2 subtype. The present invention also relates to pharmaceutically acceptable salts prepared from these compounds.

The present invention includes pharmaceutical compositions comprising a compound of the present invention or a pharmaceutically acceptable salt thereof. The pharmaceutical compositions of the present invention can be used for treating or preventing a wide variety of conditions or disorders, and particularly those disorders characterized by dysfunction of nicotinic cholinergic neurotransmission or the degeneration of the nicotinic cholinergic neurons.

The present invention includes a method for treating or preventing disorders and dysfunctions, such as CNS disorders and dysfunctions, and also for treating or preventing certain conditions, for example, alleviating pain and inflammation, in mammals in need of such treatment. The methods involve administering to a subject a therapeutically effective amount of a compound of the present invention, including a salt thereof, or a pharmaceutical composition that includes such compounds.

### BRIEF DESCRIPTION OF THE FIGURES

FIG. 1 is a graphical depiction of the results of Test Compound, (2S,4E)-N-methyl-5-(5-((3R)-1-(4,4,4-trifluorobutanoyl)pyrrolidin-3-yloxy)-6-chloro-3-pyridinyl)-4-penten-2-amine hemigalactarate, in a Water Maze Probe Trial. The graph illustrates the effect of Test Compound on Scopolamine induced deficit in mice on distance from platform, namely the measure of proximity to the platform location. For Training Days (Day 1-4)—Test Compound: i.g.; 25 min pretreatment and Scopolamine: s.c.; 15 min pretreatment and for Probe Trial (Day 5)—water i.g.; 25 min pretreatment; saline: s.c., 15 min pretreatment.

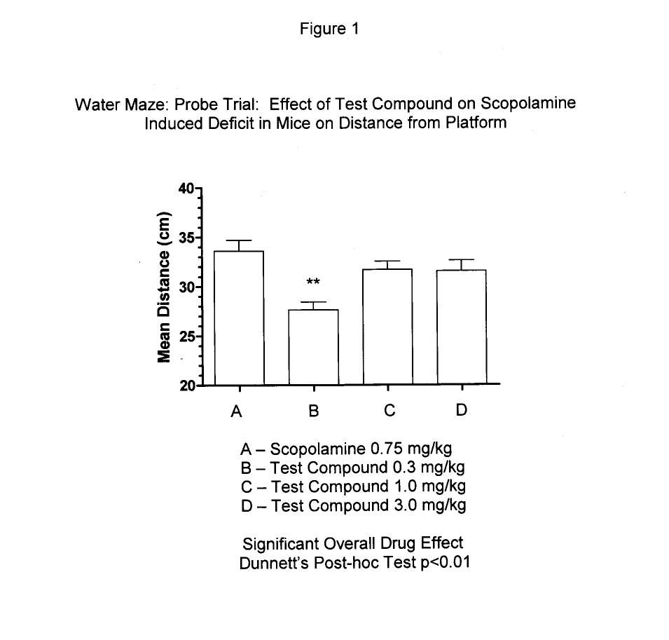

FIG. 2 is a graphical depiction of the results of Test Compound, (2S,4E)-N-methyl-5-(5-((3R)-1-(4,4,4-trifluorobutanoyl)pyrrolidin-3-yloxy)-6-chloro-3-pyridinyl)-4-penten-2-amine hemigalactarate, in a Water Maze Probe Trial. The graph illustrates the effect of Test Compound on Scopolamine induced deficit in mice on percent of time spent in the target quadrant. For Training Days (Day 1-4)—Test Compound: i.g.; 25 min pretreatment and Scopolamine: s.c.; 15 min pretreatment and for Probe Trial (Day 5)—water i.g.; 25 min pretreatment; saline: s.c., 15 min pretreatment.

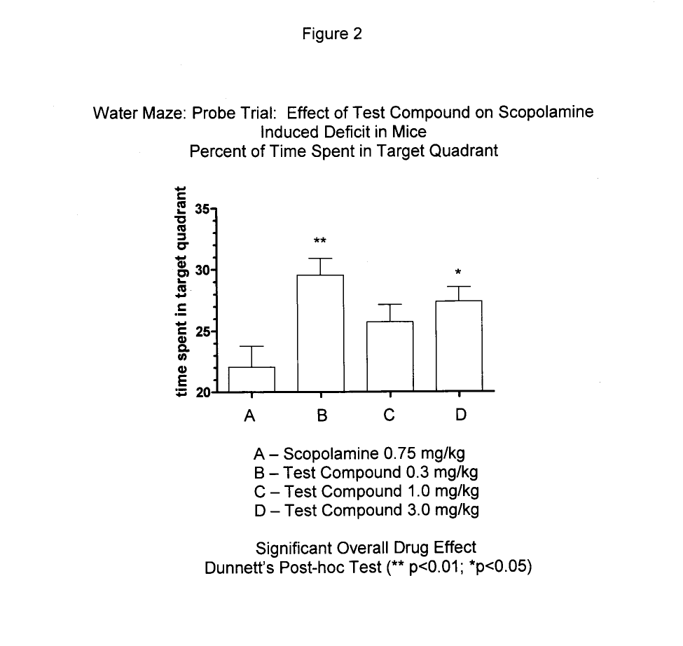

FIG. 3 is a graphical depiction of the effect of Test Compound, (2S,4E)-N-methyl-5-(5-((3R)-1-(4,4,4-trifluorobutanoyl)pyrrolidin-3-yloxy)-6-chloro-3-pyridinyl)-4-penten-2-amine hemigalactarate, (p.o.) on NOR in rats at 6 h after 3^{rd} in a series of 3 daily treatments (recall at 30 h after acclimation).

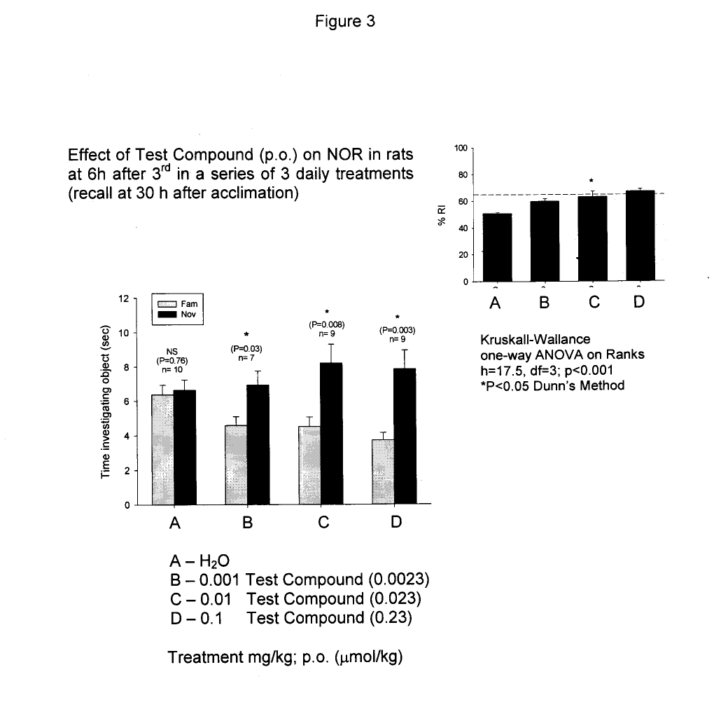

### DETAILED DESCRIPTION OF THE INVENTION

### I. Compounds

One embodiment of the present invention includes a compound as represented by Formula I:

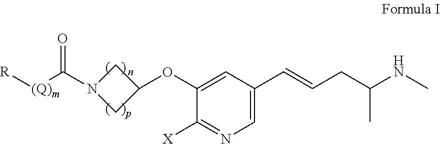

wherein:

n is 0, 1, 2, 3, or 4;

p is 0, 1, 2, 3, or 4;

and the sum of n and p is 2, 3, or 4;

m is 0 or 1;

Q is —CH₂—, —NH— or —O—;

X is H or halogen;

R is chosen from the group consisting of
C₁₋₆ alkyl, optionally substituted with one or more fluorine atoms,
C₃₋₆ cycloalkyl, optionally substituted with one or more fluorine atoms,

C₃₋₆ heterocyclyl, optionally substituted with one or more of fluorine, C₁₋₆ alkyloxy, and C₁₋₈ alkyl optionally substituted with one or more fluorine atoms,
heteroaryl, optionally substituted with one or more fluorine atoms, and

aryl, optionally substituted with one or more fluorine atoms;

or a pharmaceutically acceptable salt thereof.

In one embodiment, m is 0 and, therefore, R is bonded directly to the depicted carbonyl. In another embodiment, m is 1 and Q is —O—.

In one embodiment, a compound is selected from the group consisting of:
(2S,4E)-N-methyl-5-(5-((3R)-1-acetylpyrrolidin-3-yloxy)-3-pyridinyl)-4-penten-2-amine;
(2S,4E)-N-methyl-5-(5-((3R)-1-propanoylpyrrolidin-3-yloxy)-3-pyridinyl)-4-penten-2-amine;
(2S,4E)-N-methyl-5-(5-((3R)-1-(tetrahydro-2H-pyran-4-carbonyl)pyrrolidin-3-yloxy)-3-pyridinyl)-4-penten-2-amine;
(2S,4E)-N-methyl-5-(5-((3S)-1-acetylpyrrolidin-3-yloxy)-3-pyridinyl)-4-penten-2-amine;
(2S,4E)-N-methyl-5-(5-((3S)-1-propanoylpyrrolidin-3-yloxy)-3-pyridinyl)-4-penten-2-amine;
(2S,4E)-N-methyl-5-(5-((3S)-1-(tetrahydro-2H-pyran-4-carbonyl)pyrrolidin-3-yloxy)-3-pyridinyl)-4-penten-2-amine;
(2S,4E)-N-methyl-5-(5-(1-propanoylazetidin-3-yloxy)-3-pyridinyl)-4-penten-2-amine;
(2S,4E)-N-methyl-5-(5-(1-(tetrahydro-2H-pyran-4-carbonyl)azetidin-3-yloxy)-3-pyridinyl)-4-penten-2-amine;
(2S,4E)-N-methyl-5-(5-((3R)-1-(cyclopropylcarbonyl)pyrrolidin-3-yloxy)-3-pyridinyl)-4-penten-2-amine;
(2S,4E)-N-methyl-5-(5-((3R)-1-(methoxyacetyl)pyrrolidin-3-yloxy)-3-pyridinyl)-4-penten-2-amine;
(2S,4E)-N-methyl-5-(5-((3R)-1-(furan-2-ylcarbonyl)pyrrolidin-3-yloxy)-3-pyridinyl)-4-penten-2-amine;
(2S,4E)-N-methyl-5-(5-((3R)-1-(propoxycarbonyl)pyrrolidin-3-yloxy)-3-pyridinyl)-4-penten-2-amine;
(2S,4E)-N-methyl-5-(5-((3R)-1-(methoxyethoxycarbonyl)pyrrolidin-3-yloxy)-3-pyridinyl)-4-penten-2-amine;
(2S,4E)-N-methyl-5-(5-((3R)-1-(ethylaminocarbonyl)pyrrolidin-3-yloxy)-3-pyridinyl)-4-penten-2-amine;
(2S,4E)-N-methyl-5-(5-((3S)-1-(cyclopropylcarbonyl)pyrrolidin-3-yloxy)-3-pyridinyl)-4-penten-2-amine;
(2S,4E)-N-methyl-5-(5-((3S)-1-(methoxyacetyl)pyrrolidin-3-yloxy)-3-pyridinyl)-4-penten-2-amine;
(2S,4E)-N-methyl-5-(5-((3S)-1-(furan-2-yl-carbonyl)pyrrolidin-3-yloxy)-3-pyridinyl)-4-penten-2-amine;
(2S,4E)-N-methyl-5-(5-((3S)-1-(propoxycarbonyl)pyrrolidin-3-yloxy)-3-pyridinyl)-4-penten-2-amine;
(2S,4E)-N-methyl-5-(5-((3S)-1-(methoxyethoxycarbonyl)pyrrolidin-3-yloxy)-3-pyridinyl)-4-penten-2-amine;
(2S,4E)-N-methyl-5-(5-((3S)-1-(ethylaminocarbonyl)pyrrolidin-3-yloxy)-3-pyridinyl)-4-penten-2-amine;
(2S,4E)-N-methyl-5-(5-(1-(methoxyacetyl)azetidin-3-yloxy)-3-pyridinyl)-4-penten-2-amine;
(2S,4E)-N-methyl-5-(5-(1-(furan-2-yl-carbonyl)azetidin-3-yloxy)-3-pyridinyl)-4-penten-2-amine;
(2S,4E)-N-methyl-5-(5-(1-(propoxycarbonyl)azetidin-3-yloxy)-3-pyridinyl)-4-penten-2-amine;
(2S,4E)-N-methyl-5-(5-(1-(methoxyethoxycarbonyl)azetidin-3-yloxy)-3-pyridinyl)-4-penten-2-amine;
(2S,4E)-N-methyl-5-(5-((3S)-1-(isopropylcarbonyl)pyrrolidin-3-yloxy)-6-chloro-3-pyridinyl)-4-penten-2-amine;
(2S,4E)-N-methyl-5-(5-((3S)-1-(2,4-difluorobenzoyl)pyrrolidin-3-yloxy)-3-pyridinyl)-4-penten-2-amine;
(2S,4E)-N-methyl-5-(5-((3S)-1-(2,4-difluorobenzoyl)pyrrolidin-3-yloxy)-6-chloro-3-pyridinyl)-4-penten-2-amine;
(2S,4E)-N-methyl-5-(5-((3S)-1-(tert-butylcarbonyl)pyrrolidin-3-yloxy)-3-pyridinyl)-4-penten-2-amine;
(2S,4E)-N-methyl-5-(5-((3S)-1-(tert-butylcarbonyl)pyrrolidin-3-yloxy)-6-chloro-3-pyridinyl)-4-penten-2-amine;
(2S,4E)-N-methyl-5-(5-((3S)-1-(cyclobutylcarbonyl)pyrrolidin-3-yloxy)-3-pyridinyl)-4-penten-2-amine;
(2S,4E)-N-methyl-5-(5-((3S)-1-(cyclopentylcarbonyl)pyrrolidin-3-yloxy)-3-pyridinyl)-4-penten-2-amine;
(2S,4E)-N-methyl-5-(5-((3S)-1-(cyclopentylcarbonyl)pyrrolidin-3-yloxy)-6-chloro-3-pyridinyl)-4-penten-2-amine;
(2S,4E)-N-methyl-5-(5-((3S)-1-(tert-butylacetyl)pyrrolidin-3-yloxy)-3-pyridinyl)-4-penten-2-amine;
(2S,4E)-N-methyl-5-(5-((3S)-1-(tert-butylacetyl)pyrrolidin-3-yloxy)-6-chloro-3-pyridinyl)-4-penten-2-amine;
(2S,4E)-N-methyl-5-(5-((3S)-1-((3,3,3-trifluoropropanoyl)pyrrolidin-3-yloxy)-3-pyridinyl)-4-penten-2-amine;
(2S,4E)-N-methyl-5-(5-((3S)-1-(cyclopentylacetyl)pyrrolidin-3-yloxy)-3-pyridinyl)-4-penten-2-amine;
(2S,4E)-N-methyl-5-(5-((3S)-1-(cyclopentylacetyl)pyrrolidin-3-yloxy)-6-chloro-3-pyridinyl)-4-penten-2-amine;
(2S,4E)-N-methyl-5-(5-((3S)-1-(4,4,4-trifluorobutanoyl)pyrrolidin-3-yloxy)-3-pyridinyl)-4-penten-2-amine;
(2S,4E)-N-methyl-5-(5-((3S)-1-(4-fluorobenzoyl)pyrrolidin-3-yloxy)-3-pyridinyl)-4-penten-2-amine;
(2S,4E)-N-methyl-5-(5-((3S)-1-(4-fluorobenzoyl)pyrrolidin-3-yloxy)-6-chloro-3-pyridinyl)-4-penten-2-amine;
(2S,4E)-N-methyl-5-(5-((3R)-1-(isopropylcarbonyl)pyrrolidin-3-yloxy)-3-pyridinyl)-4-penten-2-amine;
(2S,4E)-N-methyl-5-(5-((3R)-1-(2,4-difluorobenzoyl)pyrrolidin-3-yloxy)-3-pyridinyl)-4-penten-2-amine;
(2S,4E)-N-methyl-5-(5-((3R)-1-(tert-butylcarbonyl)pyrrolidin-3-yloxy)-3-pyridinyl)-4-penten-2-amine;
(2S,4E)-N-methyl-5-(5-((3R)-1-(cyclobutylcarbonyl)pyrrolidin-3-yloxy)-3-pyridinyl)-4-penten-2-amine;
(2S,4E)-N-methyl-5-(5-((3R)-1-(cyclopentylcarbonyl)pyrrolidin-3-yloxy)-3-pyridinyl)-4-penten-2-amine;
(2S,4E)-N-methyl-5-(5-((3R)-1-(tert-butylacetyl)pyrrolidin-3-yloxy)-3-pyridinyl)-4-penten-2-amine;
(2S,4E)-N-methyl-5-(5-((3R)-1-(cyclopentylacetyl)pyrrolidin-3-yloxy)-3-pyridinyl)-4-penten-2-amine;
(2S,4E)-N-methyl-5-(5-((3R)-1-((4,4,4-trifluorobutanoyl)pyrrolidin-3-yloxy)-3-pyridinyl)-4-penten-2-amine;
(2S,4E)-N-methyl-5-(5-((3R)-1-(4-fluorobenzoyl)pyrrolidin-3-yloxy)-3-pyridinyl)-4-penten-2-amine;
(2S,4E)-N-methyl-5-(5-((3R)-1-(isopropylcarbonyl)pyrrolidin-3-yloxy)-6-chloro-3-pyridinyl)-4-penten-2-amine;
(2S,4E)-N-methyl-5-(5-((3R)-1-(2,4-difluorobenzoyl)pyrrolidin-3-yloxy)-6-chloro-3-pyridinyl)-4-penten-2-amine;
(2S,4E)-N-methyl-5-(5-((3R)-1-(tert-butylcarbonyl)pyrrolidin-3-yloxy)-6-chloro-3-pyridinyl)-4-penten-2-amine;
(2S,4E)-N-methyl-5-(5-((3R)-1-(cyclobutylcarbonyl)pyrrolidin-3-yloxy)-6-chloro-3-pyridinyl)-4-penten-2-amine;
(2S,4E)-N-methyl-5-(5-((3R)-1-(cyclopentylcarbonyl)pyrrolidin-3-yloxy)-6-chloro-3-pyridinyl)-4-penten-2-amine;
(2S,4E)-N-methyl-5-(5-((3R)-1-(tert-butylacetyl)pyrrolidin-3-yloxy)-6-chloro-3-pyridinyl)-4-penten-2-amine;
(2S,4E)-N-methyl-5-(5-((3R)-1-(3,3,3-trifluoropropanoyl)pyrrolidin-3-yloxy)-6-chloro-3-pyridinyl)-4-penten-2-amine;
(2S,4E)-N-methyl-5-(5-((3R)-1-(cyclopentylacetyl)pyrrolidin-3-yloxy)-6-chloro-3-pyridinyl)-4-penten-2-amine;
(2S,4E)-N-methyl-5-(5-((3S)-1-(isopropylcarbonyl)pyrrolidin-3-yloxy)-3-pyridinyl)-4-penten-2-amine;
(2S,4E)-N-methyl-5-(5-((3R)-1-(4-fluorobenzoyl)pyrrolidin-3-yloxy)-6-chloro-3-pyridinyl)-4-penten-2-amine;
   or a pharmaceutically acceptable salt thereof.

In one embodiment, a compound is selected from the group consisting of:
N-methyl-5-(5-(1-(4,4,4-trifluorobutanoyl)pyrrolidin-3-yloxy)-6-chloro-3-pyridinyl)-4-penten-2-amine;
(2S,4E)-N-methyl-5-(5-((3S)-1-(4,4,4-trifluorobutanoyl)pyrrolidin-3-yloxy)-6-chloro-3-pyridinyl)-4-penten-2-amine; and
(2S,4E)-N-methyl-5-(5-((3R)-1-(4,4,4-trifluorobutanoyl)pyrrolidin-3-yloxy)-6-chloro-3-pyridinyl)-4-penten-2-amine,
   or a pharmaceutically acceptable salt thereof.

One aspect of the present invention includes a pharmaceutical composition comprising a compound of the present invention and a pharmaceutically acceptable carrier.

One aspect of the present invention includes a method for the treatment or prevention of a disease or condition mediated by a neuronal nicotinic receptor comprising the administration of a compound of the present invention. In one embodiment, the neuronal nicotinic receptor is of the α4β2 subtype. In one embodiment, the disease or condition is a CNS disorder. In another embodiment, the disease or condition is one that impairs or adversely affects cognition, attention, or learning. In another embodiment, the disease or condition is pain. In another embodiment, the disease or condition is schizophrenia. In another embodiment, the disease or condition is another disorder described herein.

One aspect of the present invention includes use of a compound of the present invention for the preparation of a medicament for the treatment or prevention of a disease or condition mediated by a neuronal nicotinic receptor. In one embodiment, the neuronal nicotinic receptor is of the α4β2 subtype. In one embodiment, the disease or condition is a CNS disorder. In another embodiment, the disease or condition is one that impairs or adversely affects cognition, attention, or learning. In another embodiment, the disease or condition is pain. In another embodiment, the disease or condition is schizophrenia. In another embodiment, the disease or condition is another disorder described herein.

One aspect of the present invention includes a compound of the present invention for use as an active therapeutic substance. One aspect, thus, includes a compound of the present invention for use in the treatment or prevention of a disease or condition mediated by a neuronal nicotinic receptor. In one embodiment, the neuronal nicotinic receptor is of the α4β2 subtype. In one embodiment, the disease or condition is a CNS disorder. In another embodiment, the disease or condition is one that impairs or adversely affects cognition, attention, or learning. In another embodiment, the disease or condition is pain. In another embodiment, the disease or condition is schizophrenia. In another embodiment, the disease or condition is another disorder described herein.

The scope of the present invention includes all combinations of aspects and embodiments.

The following definitions are meant to clarify, but not limit, the terms defined. If a particular term used herein is not specifically defined, such term should not be considered indefinite. Rather, terms are used within their accepted meanings.

As used throughout this specification, the preferred number of atoms, such as carbon atoms, will be represented by, for example, the phrase “C_{x-y} alkyl,” which refers to an alkyl group, as herein defined, containing the specified number of carbon atoms. Similar terminology will apply for other preferred terms and ranges as well. Thus, for example, C₁₋₆ alkyl represents a straight or branched chain hydrocarbon containing one to six carbon atoms.

As used herein the term “alkyl” refers to a straight or branched chain hydrocarbon, which may be optionally substituted, with multiple degrees of substitution being allowed. Examples of “alkyl” as used herein include, but are not limited to, methyl, ethyl, propyl, isopropyl, isobutyl, n-butyl, tert-butyl, isopentyl, and n-pentyl.

As used herein, the term “cycloalkyl” refers to a fully saturated optionally substituted monocyclic, bicyclic, or bridged hydrocarbon ring, with multiple degrees of substitution being allowed. Exemplary “cycloalkyl” groups as used herein include, but are not limited to, cyclopropyl, cyclobutyl, cyclopentyl, cyclohexyl, and cycloheptyl.

As used herein, the term “heterocycle” or “heterocyclyl” refers to an optionally substituted mono- or polycyclic ring system, optionally containing one or more degrees of unsaturation, and also containing one or more heteroatoms, which may be optionally substituted, with multiple degrees of substitution being allowed. Exemplary heteroatoms include nitrogen, oxygen, or sulfur atoms, including N-oxides, sulfur oxides, and dioxides. Preferably, the ring is three to twelve-membered, preferably three- to eight-membered and is either fully saturated or has one or more degrees of unsaturation. Such rings may be optionally fused to one or more of another heterocyclic ring(s) or cycloalkyl ring(s). Examples of “heterocyclic” groups as used herein include, but are not limited to, tetrahydrofuran, pyran, tetrahydropyran, 1,4-dioxane, 1,3-dioxane, piperidine, pyrrolidine, morpholine, tetrahydrothiopyran, and tetrahydrothiophene.

As used herein, the term “aryl” refers to a single benzene ring or fused benzene ring system which may be optionally substituted, with multiple degrees of substitution being allowed. Examples of “aryl” groups as used include, but are not limited to, phenyl, 2-naphthyl, 1-naphthyl, anthracene, and phenanthrene. Preferable aryl rings have five- to ten-members.

As used herein, a fused benzene ring system encompassed within the term “aryl” includes fused polycyclic hydrocarbons, namely where a cyclic hydrocarbon with less than maximum number of noncumulative double bonds, for example where a saturated hydrocarbon ring (cycloalkyl, such as a cyclopentyl ring) is fused with an aromatic ring (aryl, such as a benzene ring) to form, for example, groups such as indanyl and acenaphthalenyl, and also includes such groups as, for non-limiting examples, dihydronaphthalene and tetrahydronaphthalene.

As used herein, the term “heteroaryl” refers to a monocyclic five to seven membered aromatic ring, or to a fused bicyclic aromatic ring system comprising two of such aromatic rings, which may be optionally substituted, with multiple degrees of substitution being allowed. Preferably, such rings contain five- to ten-members. These heteroaryl rings contain one or more nitrogen, sulfur, and/or oxygen atoms, where N-oxides, sulfur oxides, and dioxides are permissible heteroatom substitutions. Examples of “heteroaryl” groups as used herein include, but are not limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, quinoxaline, benzofuran, benzoxazole, benzothiophene, indole, indazole, benzimidazole, imidazopyridine, pyrazolopyridine, and pyrazolopyrimidine.

As used herein, multiple degrees of substitution includes substitution with one or more alkyl, halo, haloalkyl, alkoxy, alkylthio, aryloxy, arylthio, —NR^{a}R^{b}, —C(═O)NR^{a}R^{b}, —NR^{a}C(═O)R^{b}, —C(═O)R^{a}, —C(═O)OR^{a}, —OC(═O)R^{a}, —O(CR^{a}R^{b})₁₋₆C(═O)R^{a}, —O(CR^{a}R^{b})_{d}NR^{b}C(═O)R^{a}, —O(CR^{a}R^{b})₁₋₆NR^{b}SO₂R^{a}, —OC(═O)NR^{a}R^{b}, —NR^{a}C(═O)OR^{b}, —SO₂R^{a}, —SO₂NR^{a}R^{b}, or —NR²SO₂R³; where each R^{a} and R^{b} individually is hydrogen, alkyl, cycloalkyl, heterocyclyl, aryl, or arylalkyl, or R^{a} and R^{b} can combine with the atoms to which they are attached to form a 3- to 10-membered ring.

As used herein the terms “halo” or “halogen” refer to fluorine, chlorine, bromine, or iodine.

As used herein the term “haloalkyl” refers to an alkyl group, as defined herein, that is substituted with at least one halogen. Examples of branched or straight chained “haloalkyl” groups as used herein include, but are not limited to, methyl, ethyl, propyl, isopropyl, n-butyl, and t-butyl substituted independently with one or more halogens, for example, fluoro, chloro, bromo, and iodo. The term “haloalkyl” should be interpreted to include such substituents as perfluoroalkyl groups such as —CF₃.

As used herein the term “alkoxy” refers to a group —OR^{a}, where R^{a} is alkyl as herein defined. Likewise, the term “alkylthio” refers to a group —SR^{a}, where R^{a} is alkyl as herein defined.

As used herein the term “aryloxy” refers to a group —OR^{a}, where R^{a} is aryl as herein defined. Likewise, the term “arylthio” refers to a group —SR^{a}, where R^{a} is aryl as herein defined.

As used herein “amino” refers to a group —NR^{a}R^{b}, where each of R^{a} and R^{b} is hydrogen. Additionally, “substituted amino” refers to a group —NR^{a}R^{b} wherein each of R^{a} and R^{b} individually is alkyl, alkenyl, alkynyl, cycloalkyl, aryl, heterocylcyl, or heteroaryl. As used herein, when either R^{a} or R^{b} is other than hydrogen, such a group may be referred to as a “substituted amino” or, for example if R^{a} is H and R^{b} is alkyl, as an “alkylamino.”

As used herein, the term “pharmaceutically acceptable” refers to carrier(s), diluent(s), excipient(s) or salt forms of the compounds of the present invention that are compatible with the other ingredients of the formulation and not deleterious to the recipient of the pharmaceutical composition.

As used herein, the term “pharmaceutical composition” refers to a compound of the present invention optionally admixed with one or more pharmaceutically acceptable carriers, diluents, or exipients. Pharmaceutical compositions preferably exhibit a degree of stability to environmental conditions so as to make them suitable for manufacturing and commercialization purposes.

As used herein, the terms “effective amount”, “therapeutic amount”, and “effective dose” refer to an amount of the compound of the present invention sufficient to elicit the desired pharmacological or therapeutic effects, thus resulting in an effective treatment of a disorder. Treatment of a disorder may be manifested by delaying or preventing the onset or progression of the disorder, as well as the onset or progression of symptoms associated with the disorder. Treatment of a disorder may also be manifested by a decrease or elimination of symptoms, reversal of the progression of the disorder, as well as any other contribution to the well being of the patient.

The effective dose can vary, depending upon factors such as the condition of the patient, the severity of the symptoms of the disorder, and the manner in which the pharmaceutical composition is administered. Typically, to be administered in an effective dose, compounds may be administered in an amount of less than 5 mg/kg of patient weight. The compounds may be administered in an amount from less than about 1 mg/kg patient weight to less than about 100 μg/kg of patient weight, and further between about 1 μg/kg to less than 100 μg/kg of patient weight. The foregoing effective doses typically represent that amount that may be administered as a single dose, or as one or more doses that may be administered over a 24 hours period.

The compounds of this invention may be made by a variety of methods, including well-established synthetic methods. Illustrative general synthetic methods are set out below and then specific compounds of the invention are prepared in the working Examples.

In the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1999) Protecting Groups in Organic Synthesis, 3^{rd} Edition, John Wiley & Sons, herein incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of the present invention.

The present invention also provides a method for the synthesis of compounds useful as intermediates in the preparation of compounds of the present invention along with methods for their preparation.

The compounds can be prepared according to the methods described below using readily available starting materials and reagents. In these reactions, variants may be employed which are themselves known to those of ordinary skill in this art but are not described in detail here.

Unless otherwise stated, structures depicted herein are also meant to include compounds which differ only in the presence of one or more isotopically enriched atoms. Compounds having the present structure except for the replacement of a hydrogen atom by a deuterium or tritium, or the replacement of a carbon atom by a ¹³C- or ¹⁴C-enriched carbon are within the scope of the invention. For example, deuterium has been widely used to examine the pharmacokinetics and metabolism of biologically active compounds. Although deuterium behaves similarly to hydrogen from a chemical perspective, there are significant differences in bond energies and bond lengths between a deuterium-carbon bond and a hydrogen-carbon bond. Consequently, replacement of hydrogen by deuterium in a biologically active compound may result in a compound that generally retains its biochemical potency and selectivity but manifests significantly different absorption, distribution, metabolism, and/or excretion (ADME) properties compared to its isotope-free counterpart. Thus, deuterium substitution may result in improved drug efficacy, safety, and/or tolerability for some biologically active compounds.

The compounds of the present invention may crystallize in more than one form, a characteristic known as polymorphism, and such polymorphic forms (“polymorphs”) are within the scope of the present invention. Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by the formulae of the present invention, as well as any wholly or partially equilibrated mixtures thereof. The present invention also includes the individual isomers of the compounds represented by the formulas above as mixtures with isomers thereof in which one or more chiral centers are inverted.

When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be effected by any suitable method known in the art. See, for example, Stereochemistry of Organic Compounds (Wiley-Interscience, 1994).

The present invention includes a salt or solvate of the compounds herein described, including combinations thereof such as a solvate of a salt. The compounds of the present invention may exist in solvated, for example hydrated, as well as unsolvated forms, and the present invention encompasses all such forms.

Typically, but not absolutely, the salts of the present invention are pharmaceutically acceptable salts. Salts encompassed within the term “pharmaceutically acceptable salts” refer to non-toxic salts of the compounds of this invention.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

### II. General Synthetic Methods

The general strategy for the synthesis of compounds of the present invention is illustrated in Scheme 1. Briefly, either 3-bromo-5-hydroxypyridine (1a) or an appropriately 6-substituted-3-bromo-5-hydroxypyridine (such as 1b) can be coupled, using Mitsunobu or similar conditions, with an N-protected azetidinol, pyrrolidinol or piperidinol. The tert-butoxycarbonyl protecting group is typically employed to protect the amine nitrogen. Subsequent removal of the tert-butoxycarbonyl protecting group from the coupling product provides compound 2. Reaction of 2 with various reactive carboxylic acid, carbonic acid and carbamic acid equivalents yields compound 3, where R and Q are as defined previously. Heck, or other similar, coupling of this compound with N-tert-butoxycarbonyl-N-methyl-4-penten-2-amine then gives 4, which when subsequently deprotected, provides compound 5, a compound of the present invention. Palladium catalysis is typically employed for such couplings between aryl halides and alkenes.

Examples of reagent classes that can be used to convert compound 2 into compound 3 include acid chlorides (including chloroformates), carbonic esters and isocyanates, giving rise to carboxamide, carbamate (urethane) and urea versions of 3, all of which can be carried forward as described to yield compounds of the present invention.

Variations in the strategy shown in Scheme 1 are possible. For instance, the Heck coupling can be performed before the Mitsunobu coupling. That is, compound 1 can be coupled with N-tert-butoxycarbonyl-N-methyl-4-penten-2-amine in a palladium catalyzed process, and the intermediate thus produced can be coupled with the protected azetidinol, pyrrolidinol, or piperidinol to generate compound 4.

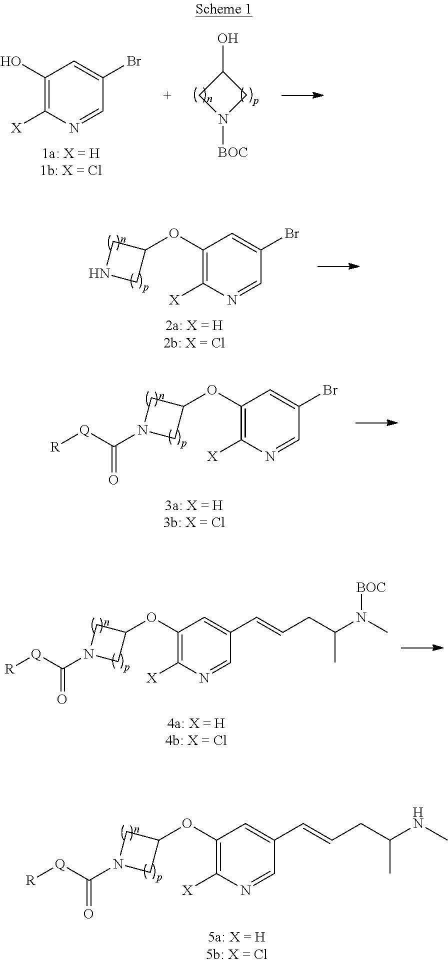

Those skilled in the art will recognize that numerous protecting groups and protection/deprotection schemes are available to achieve results comparable to those illustrated here. (See, for example, T. W. Green and P. G. M. Wuts (1999) Protecting Groups in Organic Synthesis, 3^{rd} Edition, John Wiley & Sons). For instance, a different protecting group can be used for the N-methyl-4-penten-2-amine portion as compared to the portion derived from the azetidinol, pyrrolidinol or piperidinol. This would allow for introduction of the acyl group at the azetidinol, pyrrolidinol or piperidinol nitrogen as the penultimate step in the sequence.

Those skilled in the art of organic synthesis will also appreciate that there exist multiple means of producing compounds of the present invention, as well as means for producing compounds of the present invention which are labeled with a radioisotope appropriate to various uses. For example, use of an appropriate ¹¹C- or ¹⁸F-labeled activated carboxylic acid in the reaction sequences described above will produce the corresponding ¹¹C- or ¹⁸F-labeled compound for use in positron emission tomography. Likewise, use of a ³H or ¹⁴C-labeled activated carboxylic acid in the reaction sequences described above will produce isotopically modified compounds suitable for use in receptor binding and metabolism studies or as alternative therapeutic compounds.

### III. Pharmaceutical Compositions

Although it is possible to administer the compound of the present invention in the form of a bulk active chemical, it is preferred to administer the compound in the form of a pharmaceutical composition or formulation. Thus, one aspect the present invention includes pharmaceutical compositions comprising one or more compounds of Formula I and/or pharmaceutically acceptable salts thereof and one or more pharmaceutically acceptable carriers, diluents, or excipients. Another aspect of the invention provides a process for the preparation of a pharmaceutical composition including admixing one or more compounds of Formula I and/or pharmaceutically acceptable salts thereof with one or more pharmaceutically acceptable carriers, diluents or excipients.

The manner in which the compound of the present invention is administered can vary. The compound of the present invention is preferably administered orally. Preferred pharmaceutical compositions for oral administration include tablets, capsules, caplets, syrups, solutions, and suspensions. The pharmaceutical compositions of the present invention may be provided in modified release dosage forms such as time-release tablet and capsule formulations.

The pharmaceutical compositions can also be administered via injection, namely, intravenously, intramuscularly, subcutaneously, intraperitoneally, intraarterially, intrathecally, and intracerebroventricularly. Intravenous administration is a preferred method of injection. Suitable carriers for injection are well known to those of skill in the art and include 5% dextrose solutions, saline, and phosphate buffered saline.

The formulations may also be administered using other means, for example, rectal administration. Formulations useful for rectal administration, such as suppositories, are well known to those of skill in the art. The compounds can also be administered by inhalation, for example, in the form of an aerosol; topically, such as, in lotion form; transdermally, such as, using a transdermal patch (for example, by using technology that is commercially available from Novartis and Alza Corporation), by powder injection, or by buccal, sublingual, or intranasal absorption.

Pharmaceutical compositions may be formulated in unit dose form, or in multiple or subunit doses

The administration of the pharmaceutical compositions described herein can be intermittent, or at a gradual, continuous, constant or controlled rate. The pharmaceutical compositions may be administered to a warm-blooded animal, for example, a mammal such as a mouse, rat, cat, rabbit, dog, pig, cow, or monkey; but advantageously is administered to a human being. In addition, the time of day and the number of times per day that the pharmaceutical composition is administered can vary.

The compounds of the present invention may be used in the treatment of a variety of disorders and conditions and, as such, may be used in combination with a variety of other suitable therapeutic agents useful in the treatment or prophylaxis of those disorders or conditions. Thus, one embodiment of the present invention includes the administration of the compound of the present invention in combination with other therapeutic compounds. For example, the compound of the present invention can be used in combination with other NNR ligands (such as varenicline), allosteric modulators of NNRs, antioxidants (such as free radical scavenging agents), antibacterial agents (such as penicillin antibiotics), antiviral agents (such as nucleoside analogs, like zidovudine and acyclovir), anticoagulants (such as warfarin), anti-inflammatory agents (such as NSAIDs), anti-pyretics, analgesics, anesthetics (such as used in surgery), acetylcholinesterase inhibitors (such as donepezil and galantamine), antipsychotics (such as haloperidol, clozapine, olanzapine, and quetiapine), immuno-suppressants (such as cyclosporin and methotrexate), neuroprotective agents, steroids (such as steroid hormones), corticosteroids (such as dexamethasone, predisone, and hydrocortisone), vitamins, minerals, nutraceuticals, anti-depressants (such as imipramine, fluoxetine, paroxetine, escitalopram, sertraline, venlafaxine, and duloxetine), anxiolytics (such as alprazolam and buspirone), anticonvulsants (such as phenyloin and gabapentin), vasodilators (such as prazosin and sildenafil), mood stabilizers (such as valproate and aripiprazole), anti-cancer drugs (such as anti-proliferatives), antihypertensive agents (such as atenolol, clonidine, amlopidine, verapamil, and olmesartan), laxatives, stool softeners, diuretics (such as furosemide), anti-spasmotics (such as dicyclomine), anti-dyskinetic agents, and anti-ulcer medications (such as esomeprazole). Such a combination of pharmaceutically active agents may be administered together or separately and, when administered separately, administration may occur simultaneously or sequentially, in any order. The amounts of the compounds or agents and the relative timings of administration will be selected in order to achieve the desired therapeutic effect. The administration in combination of a compound of the present invention with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second. Such sequential administration may be close in time or remote in time.

Another aspect of the present invention includes combination therapy comprising administering to the subject a therapeutically or prophylactically effective amount of the compound of the present invention and one or more other therapy including chemotherapy, radiation therapy, gene therapy, or immunotherapy.

### IV. Method of Using Pharmaceutical Compositions

The compounds of the present invention can be used for the prevention or treatment of various conditions or disorders for which other types of nicotinic compounds have been proposed or are shown to be useful as therapeutics, such as CNS disorders, inflammation, inflammatory response associated with bacterial and/or viral infection, pain, metabolic syndrome, autoimmune disorders, addictions, obesity or other disorders described in further detail herein. This compound can also be used as a diagnostic agent in receptor binding studies (in vitro and in vivo). Such therapeutic and other teachings are described, for example, in references previously listed herein, including Williams et al., Drug News Perspec. 7(4): 205 (1994), Arneric et al., CNS Drug Rev. 1(1): 1-26 (1995), Arneric et al., Exp. Opin. Invest. Drugs 5(1): 79-100 (1996), Bencherif et al., J. Pharmacol. Exp. Ther. 279: 1413 (1996), Lippiello et al., J. Pharmacol. Exp. Ther. 279: 1422 (1996), Damaj et al., J. Pharmacol. Exp. Ther. 291: 390 (1999); Chiari et al., Anesthesiology 91: 1447 (1999), Lavand'homme and Eisenbach, Anesthesiology 91: 1455 (1999), Holladay et al., J. Med. Chem. 40(28): 4169-94 (1997), Bannon et al., Science 279: 77 (1998), PCT WO 94/08992, PCT WO 96/31475, PCT WO 96/40682, and U.S. Pat. No. 5,583,140 to Bencherif et al., U.S. Pat. No. 5,597,919 to Dull et al., U.S. Pat. No. 5,604,231 to Smith et al. and U.S. Pat. No. 5,852,041 to Cosford et al.

### CNS Disorders

The compounds and their pharmaceutical compositions are useful in the treatment or prevention of a variety of CNS disorders, including neurodegenerative disorders, neuropsychiatric disorders, neurologic disorders, and addictions. The compounds and their pharmaceutical compositions can be used to treat or prevent cognitive deficits and dysfunctions, age-related and otherwise; attentional disorders and dementias, including those due to infectious agents or metabolic disturbances; to provide neuroprotection; to treat convulsions and multiple cerebral infarcts; to treat mood disorders, compulsions and addictive behaviors; to provide analgesia; to control inflammation, such as mediated by cytokines and nuclear factor kappa B; to treat inflammatory disorders; to provide pain relief; and to treat infections, as anti-infectious agents for treating bacterial, fungal, and viral infections. Among the disorders, diseases and conditions that the compounds and pharmaceutical compositions of the present invention can be used to treat or prevent are: age-associated memory impairment (AAMI), mild cognitive impairment (MCI), age-related cognitive decline (ARCD), pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, central ischemia, peripheral ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, tardive dyskinesia, spastic dystonia, hyperkinesia, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), and chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, conduct disorder, oppositional defiant disorder, Tourette's syndrome, autism, drug and alcohol addiction, tobacco addiction, compulsive overeating and sexual dysfunction.

Cognitive impairments or dysfunctions may be associated with psychiatric disorders or conditions, such as schizophrenia and other psychotic disorders, including but not limited to psychotic disorder, schizophreniform disorder, schizoaffective disorder, delusional disorder, brief psychotic disorder, shared psychotic disorder, and psychotic disorders due to a general medical conditions, dementias and other cognitive disorders, including but not limited to mild cognitive impairment, pre-senile dementia, Alzheimer's disease, senile dementia, dementia of the Alzheimer's type, age-related memory impairment, Lewy body dementia, vascular dementia, AIDS dementia complex, dyslexia, Parkinsonism including Parkinson's disease, cognitive impairment and dementia of Parkinson's Disease, cognitive impairment of multiple sclerosis, cognitive impairment caused by traumatic brain injury, dementias due to other general medical conditions, anxiety disorders, including but not limited to panic disorder without agoraphobia, panic disorder with agoraphobia, agoraphobia without history of panic disorder, specific phobia, social phobia, obsessive-compulsive disorder, post-traumatic stress disorder, acute stress disorder, generalized anxiety disorder and generalized anxiety disorder due to a general medical condition, mood disorders, including but not limited to major depressive disorder, dysthymic disorder, bipolar depression, bipolar mania, bipolar I disorder, depression associated with manic, depressive or mixed episodes, bipolar II disorder, cyclothymic disorder, and mood disorders due to general medical conditions, sleep disorders, including but not limited to dyssomnia disorders, primary insomnia, primary hypersomnia, narcolepsy, parasomnia disorders, nightmare disorder, sleep terror disorder and sleepwalking disorder, mental retardation, learning disorders, motor skills disorders, communication disorders, pervasive developmental disorders, attention-deficit and disruptive behavior disorders, attention deficit disorder, attention deficit hyperactivity disorder, feeding and eating disorders of infancy, childhood, or adults, tic disorders, elimination disorders, substance-related disorders, including but not limited to substance dependence, substance abuse, substance intoxication, substance withdrawal, alcohol-related disorders, amphetamine or amphetamine-like-related disorders, caffeine-related disorders, cannabis-related disorders, cocaine-related disorders, hallucinogen-related disorders, inhalant-related disorders, nicotine-related disorders, opioid-related disorders, phencyclidine or phencyclidine-like-related disorders, and sedative-, hypnotic- or anxiolytic-related disorders, personality disorders, including but not limited to obsessive-compulsive personality disorder and impulse-control disorders.

Cognitive performance may be assessed with a validated cognitive scale, such as, for example, the cognitive subscale of the Alzheimer's Disease Assessment Scale (ADAS-cog). One measure of the effectiveness of the compounds of the present invention in improving cognition may include measuring a patient's degree of change according to such a scale.

Regarding compulsions and addictive behaviors, the compounds of the present invention may be used as a therapy for nicotine addiction and for other brain-reward disorders, such as substance abuse including alcohol addiction, illicit and prescription drug addiction, eating disorders, including obesity, and behavioral addictions, such as gambling, or other similar behavioral manifestations of addiction.

The above conditions and disorders are discussed in further detail, for example, in the American Psychiatric Association: Diagnostic and Statistical Manual of Mental Disorders, Fourth Edition, Text Revision, Washington, D.C., American Psychiatric Association, 2000. This Manual may also be referred to for greater detail on the symptoms and diagnostic features associated with substance use, abuse, and dependence.

Preferably, the treatment or prevention of diseases, disorders and conditions occurs without appreciable adverse side effects, including, for example, significant increases in blood pressure and heart rate, significant negative effects upon the gastro-intestinal tract, and significant effects upon skeletal muscle.

The compounds of the present invention, when employed in effective amounts, are believed to modulate the activity of the α4β2 NNR subtype without appreciable interaction with the nicotinic subtypes that characterize the human ganglia, as demonstrated by a lack of the ability to elicit nicotinic function in adrenal chromaffin tissue, or skeletal muscle, further demonstrated by a lack of the ability to elicit nicotinic function in cell preparations expressing muscle-type nicotinic receptors. Thus, these compounds are believed capable of treating or preventing diseases, disorders and conditions without eliciting significant side effects associated activity at ganglionic and neuromuscular sites. Thus, administration of the compounds is believed to provide a therapeutic window in which treatment of certain diseases, disorders and conditions is provided, and certain side effects are avoided. That is, an effective dose of the compound is believed sufficient to provide the desired effects upon the disease, disorder or condition, but is believed insufficient, namely is not at a high enough level, to provide undesirable side effects.

Thus, the present invention provides the use of a compound of the present invention, or a pharmaceutically acceptable salt thereof, for use in therapy, such as a therapy described above.

In yet another aspect the present invention provides the use of a compound of the present invention, or a pharmaceutically acceptable salt thereof, in the manufacture of a medicament for use in the treatment of a CNS disorder, such as a disorder, disease or condition described hereinabove.

### Inflammation

The nervous system, primarily through the vagus nerve, is known to regulate the magnitude of the innate immune response by inhibiting the release of macrophage tumor necrosis factor (TNF). This physiological mechanism is known as the “cholinergic anti-inflammatory pathway” (see, for example, Tracey, “The Inflammatory Reflex,” Nature 420: 853-9 (2002)). Excessive inflammation and tumor necrosis factor synthesis cause morbidity and even mortality in a variety of diseases. These diseases include, but are not limited to, endotoxemia, rheumatoid arthritis, osteoarthritis, psoriasis, asthma, atherosclerosis, idiopathic pulmonary fibrosis, and inflammatory bowel disease.

Inflammatory conditions that can be treated or prevented by administering the compounds described herein include, but are not limited to, chronic and acute inflammation, psoriasis, endotoxemia, gout, acute pseudogout, acute gouty arthritis, arthritis, rheumatoid arthritis, osteoarthritis, allograft rejection, chronic transplant rejection, asthma, atherosclerosis, mononuclear-phagocyte dependent lung injury, idiopathic pulmonary fibrosis, atopic dermatitis, chronic obstructive pulmonary disease, adult respiratory distress syndrome, acute chest syndrome in sickle cell disease, inflammatory bowel disease, irritable bowel syndrome, Crohn's disease, ulcers, ulcerative colitis, acute cholangitis, aphthous stomatitis, cachexia, pouchitis, glomerulonephritis, lupus nephritis, thrombosis, and graft vs. host reaction.

Inflammatory Response Associated with Bacterial and/or Viral Infection

Many bacterial and/or viral infections are associated with side effects brought on by the formation of toxins, and the body's natural response to the bacteria or virus and/or the toxins. As discussed above, the body's response to infection often involves generating a significant amount of TNF and/or other cytokines. The over-expression of these cytokines can result in significant injury, such as septic shock (when the bacteria is sepsis), endotoxic shock, urosepsis, viral pneumonitis and toxic shock syndrome.

Cytokine expression is mediated by NNRs, and can be inhibited by administering agonists or partial agonists of these receptors. Those compounds described herein that are agonists or partial agonists of these receptors can therefore be used to minimize the inflammatory response associated with bacterial infection, as well as viral and fungal infections. Examples of such bacterial infections include anthrax, botulism, and sepsis. Some of these compounds may also have antimicrobial properties.

These compounds can also be used as adjunct therapy in combination with existing therapies to manage bacterial, viral and fungal infections, such as antibiotics, antivirals and antifungals. Antitoxins can also be used to bind to toxins produced by the infectious agents and allow the bound toxins to pass through the body without generating an inflammatory response. Examples of antitoxins are disclosed, for example, in U.S. Pat. No. 6,310,043 to Bundle et al. Other agents effective against bacterial and other toxins can be effective and their therapeutic effect can be complemented by co-administration with the compounds described herein.

### Pain

The compounds can be administered to treat and/or prevent pain, including acute, neurologic, inflammatory, neuropathic and chronic pain. The compounds can be used in conjunction with opiates to minimize the likelihood of opiate addiction (e.g., morphine sparing therapy). The analgesic activity of compounds described herein can be demonstrated in models of persistent inflammatory pain and of neuropathic pain, performed as described in U.S. Published Patent Application No. 20010056084 A1 (Allgeier et al.) (e.g., mechanical hyperalgesia in the complete Freund's adjuvant rat model of inflammatory pain and mechanical hyperalgesia in the mouse partial sciatic nerve ligation model of neuropathic pain).

The analgesic effect is suitable for treating pain of various genesis or etiology, in particular in treating inflammatory pain and associated hyperalgesia, neuropathic pain and associated hyperalgesia, chronic pain (e.g., severe chronic pain, post-operative pain and pain associated with various conditions including cancer, angina, renal or biliary colic, menstruation, migraine, and gout). Inflammatory pain may be of diverse genesis, including arthritis and rheumatoid disease, teno-synovitis and vasculitis. Neuropathic pain includes trigeminal or herpetic neuralgia, neuropathies such as diabetic neuropathy pain, causalgia, low back pain and deafferentation syndromes such as brachial plexus avulsion.

### Neovascularization

The α7 NNR is associated with neovascularization. Inhibition of neovascularization, for example, by administering antagonists (or at certain dosages, partial agonists) of the α7 NNR can treat or prevent conditions characterized by undesirable neovascularization or angiogenesis. Such conditions can include those characterized by inflammatory angiogenesis and/or ischemia-induced angiogenesis. Neovascularization associated with tumor growth can also be inhibited by administering those compounds described herein that function as antagonists or partial agonists of α7 NNR.

Specific antagonism of α7 NNR-specific activity reduces the angiogenic response to inflammation, ischemia, and neoplasia. Guidance regarding appropriate animal model systems for evaluating the compounds described herein can be found, for example, in Heeschen, C. et al., “A novel angiogenic pathway mediated by non-neuronal nicotinic acetylcholine receptors,” J. Clin. Invest. 110(4):527-36 (2002).

Representative tumor types that can be treated using the compounds described herein include NSCLC, ovarian cancer, pancreatic cancer, breast carcinoma, colon carcinoma, rectum carcinoma, lung carcinoma, oropharynx carcinoma, hypopharynx carcinoma, esophagus carcinoma, stomach carcinoma, pancreas carcinoma, liver carcinoma, gallbladder carcinoma, bile duct carcinoma, small intestine carcinoma, urinary tract carcinoma, kidney carcinoma, bladder carcinoma, urothelium carcinoma, female genital tract carcinoma, cervix carcinoma, uterus carcinoma, ovarian carcinoma, choriocarcinoma, gestational trophoblastic disease, male genital tract carcinoma, prostate carcinoma, seminal vesicles carcinoma, testes carcinoma, germ cell tumors, endocrine gland carcinoma, thyroid carcinoma, adrenal carcinoma, pituitary gland carcinoma, skin carcinoma, hemangiomas, melanomas, sarcomas, bone and soft tissue sarcoma, Kaposi's sarcoma, tumors of the brain, tumors of the nerves, tumors of the eyes, tumors of the meninges, astrocytomas, gliomas, glioblastomas, retinoblastomas, neuromas, neuroblastomas, Schwannomas, meningiomas, solid tumors arising from hematopoietic malignancies (such as leukemias, chloromas, plasmacytomas and the plaques and tumors of mycosis fungoides and cutaneous T-cell lymphoma/leukemia), and solid tumors arising from lymphomas.

The compounds can also be administered in conjunction with other forms of anti-cancer treatment, including co-administration with antineoplastic antitumor agents such as cis-platin, adriamycin, daunomycin, and the like, and/or anti-VEGF (vascular endothelial growth factor) agents, as such are known in the art.

The compounds can be administered in such a manner that they are targeted to the tumor site. For example, the compounds can be administered in microspheres, microparticles or liposomes conjugated to various antibodies that direct the microparticles to the tumor. Additionally, the compounds can be present in microspheres, microparticles or liposomes that are appropriately sized to pass through the arteries and veins, but lodge in capillary beds surrounding tumors and administer the compounds locally to the tumor. Such drug delivery devices are known in the art.

### Other Disorders

In addition to treating CNS disorders, inflammation, and neovascularization, and pain, the compounds of the present invention can be also used to prevent or treat certain other conditions, diseases, and disorders in which NNRs play a role. Examples include autoimmune disorders such as lupus, disorders associated with cytokine release, cachexia secondary to infection (e.g., as occurs in AIDS, AIDS related complex and neoplasia), obesity, pemphitis, urinary incontinence, overactive bladder, diarrhea, constipation, retinal diseases, infectious diseases, myasthenia, Eaton-Lambert syndrome, hypertension, preeclampsia, osteoporosis, vasoconstriction, vasodilatation, cardiac arrhythmias, type I diabetes, type II diabetes, bulimia, anorexia and sexual dysfunction, as well as those indications set forth in published PCT application WO 98/25619. The compounds of this invention can also be administered to treat convulsions such as those that are symptomatic of epilepsy, and to treat conditions such as syphillis and Creutzfeld-Jakob disease.

### Diagnostic Uses

The compounds can be used in diagnostic compositions, such as probes, particularly when they are modified to include appropriate labels. The probes can be used, for example, to determine the relative number and/or function of specific receptors, particularly the α4β2 receptor subtype. For this purpose the compounds of the present invention most preferably are labeled with a radioactive isotopic moiety such as ¹¹C, ¹⁸F, ⁷⁶Br, ¹²³I or ¹²⁵I.

The administered compounds can be detected using known detection methods appropriate for the label used. Examples of detection methods include position emission topography (PET) and single-photon emission computed tomography (SPECT). The radiolabels described above are useful in PET (e.g., ¹¹C, ¹⁸F or ⁷⁶Br) and SPECT (e.g., ¹²³I) imaging, with half-lives of about 20.4 minutes for ¹¹C, about 109 minutes for ¹⁸F, about 13 hours for ¹²³I, and about 16 hours for ⁷⁶Br. A high specific activity is desired to visualize the selected receptor subtypes at non-saturating concentrations. The administered doses typically are below the toxic range and provide high contrast images. The compounds are expected to be capable of administration in non-toxic levels. Determination of dose is carried out in a manner known to one skilled in the art of radiolabel imaging. See, for example, U.S. Pat. No. 5,969,144 to London et al.

The compounds can be administered using known techniques. See, for example, U.S. Pat. No. 5,969,144 to London et al., as noted. The compounds can be administered in formulation compositions that incorporate other ingredients, such as those types of ingredients that are useful in formulating a diagnostic composition. Compounds useful in accordance with carrying out the present invention most preferably are employed in forms of high purity. See, U.S. Pat. No. 5,853,696 to Elmalch et al.,

After the compounds are administered to a subject (e.g., a human subject), the presence of that compound within the subject can be imaged and quantified by appropriate techniques in order to indicate the presence, quantity, and functionality of selected NNR subtypes. In addition to humans, the compounds can also be administered to animals, such as mice, rats, dogs, and monkeys. SPECT and PET imaging can be carried out using any appropriate technique and apparatus. See Villemagne et al., In: Arneric et al. (Eds.) Neuronal Nicotinic Receptors: Pharmacology and Therapeutic Opportunities, 235-250 (1998) and U.S. Pat. No. 5,853,696 to Elmalch et al., each herein incorporated by reference, for a disclosure of representative imaging techniques.

The radiolabeled compounds bind with high affinity to selective NNR subtypes (e.g., α4β2) and preferably exhibit negligible non-specific binding to other nicotinic cholinergic receptor subtypes (e.g., those receptor subtypes associated with muscle and ganglia). As such, the compounds can be used as agents for noninvasive imaging of nicotinic cholinergic receptor subtypes within the body of a subject, particularly within the brain for diagnosis associated with a variety of CNS diseases and disorders.

In one aspect, the diagnostic compositions can be used in a method to diagnose disease in a subject, such as a human patient. The method involves administering to that patient a detectably labeled compound as described herein, and detecting the binding of that compound to selected NNR subtypes (e.g., α4β2 receptor subtype). Those skilled in the art of using diagnostic tools, such as PET and SPECT, can use the radiolabeled compounds described herein to diagnose a wide variety of conditions and disorders, including conditions and disorders associated with dysfunction of the central and autonomic nervous systems. Such disorders include a wide variety of CNS diseases and disorders, including Alzheimer's disease, Parkinson's disease, and schizophrenia. These and other representative diseases and disorders that can be evaluated include those that are set forth in U.S. Pat. No. 5,952,339 to Bencherif et al.

In another aspect, the diagnostic compositions can be used in a method to monitor selective nicotinic receptor subtypes of a subject, such as a human patient. The method involves administering a detectably labeled compound as described herein to that patient and detecting the binding of that compound to selected nicotinic receptor subtypes namely, the β4β2 receptor subtype.

### Receptor Binding

The compounds of this invention can be used as reference ligands in binding assays for compounds which bind to NNR subtypes, particularly the α4β2 receptor subtype. For this purpose the compounds of this invention are preferably labeled with a radioactive isotopic moiety such as ³H, or ¹⁴C. Examples of such binding assays are described in detail below.

### IV. Synthetic Examples

### Example 1

### (R)-5-(pyrrolidin-3-yloxy)-3-bromopyridine

To a solution of (S)—N-tert-butoxycarbonyl-3-hydroxypyrrolidine (6.92 g, 37.0 mmol), 3-bromo-5-hydroxypyridine (5.80 g, 33.7 mmol) and triphenylphosphine (10.6 g, 40.4 mmol) in dichloromethane:1,4-dioxane (100 mL, 3:2) was added diethyl azodicarboxylate (17.6 mL, 40% solution in toluene, 40.4 mmol). The solution was stirred at ambient temperature for 16 h. Trifluoroacetic acid (100 mL) was added and the solution was stirred at ambient temperature for 24 h. The solvent was evaporated and the residue was dissolved methanol (100 mL). Amberlyst A26 (OH⁻) (15 g) was added and the mixture was stirred for 15 min. The mixture was then filtered, and filtrate was stirred with Dowex 50 WX8-200 resin (15 g) for 15 min. The mixture was filtered, and resin was washed with methanol (100 mL). The filtrates were discarded, and the resin was eluted with a 7M ammonia in methanol solution (200 mL). The eluent was concentrated to obtain (R)-5-(pyrrolidin-3-yloxy)-3-bromopyridine (6.2 g, 76% yield) as colorless gum.

### Example 2

### (R)-5-(1-(tert-butylacetyl)pyrrolidin-3-yloxy)-3-bromopyridine

To a solution of (R)-5-(pyrrolidin-3-yloxy)-3-bromopyridine (0.600 g, 2.46 mmol) and triethylamine (1.30 mL, 9.87 mmol) in dichloromethane (10 mL) at 0° C. was added tert-butylacetyl chloride (0.43 g, 3.2 mmol). The solution was stirred at ambient temperature for 1 h. The solution was diluted with dichloromethane (30 mL), washed with saturated aqueous sodium bicarbonate solution (30 mL), concentrated, and purified on HPLC, using mixtures of 0.05% trifluoroacetic acid (TFA) in water and 0.05% TFA in acetonitrile, to obtain (R)-5-(1-(tert-butylacetyl)pyrrolidin-3-yloxy)-3-bromopyridine trifluoroacetate. This was then dissolved in methanol (20 mL) and stirred with Amberlyst A26 (OH⁻) (5 g) for 10 min and filtered. The filtrate was concentrated to obtain (R)-5-(1-(tert-butylacetyl)pyrrolidin-3-yloxy)-3-bromopyridine (0.4 g) as white solid. ¹H NMR (CDCl₃, 400 MHz): δ 8.34-8.31 (m, 1H), 8.22-8.19 (m, 1H), 7.36-7.33 (m, 1H), 5.04-4.92 (m, 1H), 3.86-3.58 (m, 4H), 2.33-2.10 (m, 4H), 1.08 and 1.06 (s, 9H).

### Example 3

### (2S,4E)-N-methyl-5-(5-((3R)-1-(tert-butylacetyl)pyrrolidin-3-yloxy)-3-pyridinyl)-4-penten-2-amine

A mixture of (S)—N-tert-butoxycarbonyl-N-methyl-4-penten-2-amine (0.050 g, 0.25 mmol), (R)-5-(1-(tert-butylacetyl)pyrrolidin-3-yloxy)-3-bromopyridine (0.110 g, 0.325 mmol), palladium acetate (3.0 mg, 0.013 mmol), tricyclohexylphosphine (7.0 mg, 0.025 mmol) and diisopropylethylamine (0.18 mL, 1.0 mmol) in N-methylpyrrolidinone (3 mL) was stirred at 130° C. for 15 h. The reaction was diluted with 3 mL of ethyl acetate and washed with 5 mL of water. The organic layer was concentrated and purified on HPLC, using mixtures of 0.05% TFA in water and 0.05% TFA in acetonitrile, to obtain (2S,4E)-N-tert-butoxycarbonyl-N-methyl-5-(5-((3R)-1-(tert-butylacetyl)pyrrolidin-3-yloxy)-3-pyridinyl)-4-penten-2-amine as gum. This was dissolved in dichloromethane (3 mL) and treated with trifluoroacetic acid (2 mL) for 2 h. The solvent was evaporated, and the residue was purified by HPLC using 0.05% TFA in water and 0.05% TFA in acetonitrile to obtain (2S,4E)-N-methyl-5-(5-((3R)-1-(tert-butylacetyl)pyrrolidin-3-yloxy)-3-pyridinyl)-4-penten-2-amine trifluoroacetate (0.070 g, 78% yield) as gum. ¹H NMR (CD₃OD, 400 MHz): δ 8.39 (d, J=1.5 Hz, 1H), 8.34-8.32 (m, 1H), 7.91 (bs, 1H), 6.71 (d, J=16.1 Hz, 1H), 6.60-6.53 (m, 1H), 5.30-5.25 (m, 1H), 3.88-3.67 (m, 4H), 3.45-3.38 (m, 1H), 2.78-2.70 (m, 4H), 2.62-2.54 (m, 1H), 2.37-2.21 (m, 4H), 1.37 (d, J=6.6 Hz, 3H), 1.06 and 1.04 (s, 9H);

LCMS (m/z): 360 (M+1).

### Example 4

### (R)-3-bromo-5-(pyrrolidin-3-yloxy)-6-chloropyridine

To a solution of (S)—N-tert-butoxycarbonyl-3-hydroxypyrrolidine (4.85 g, 25.9 mmol), 3-bromo-5-hydroxy-6-chloropyridine (4.50 g, 21.6 mmol) and triphenylphosphine (6.80 g, 25.9 mmol) in dichloromethane (200 mL) was added diethyl azodicarboxylate (10.0 mL, 40% solution in toluene, 25.9 mmol). The solution was stirred at ambient temperature for 18 h. The solvent was evaporated, and the residue was purified on a silica gel column, using 40% ethyl acetate in hexanes, to obtain (R)—N-tert-butoxycarbonyl-3-bromo-5-(pyrrolidin-3-yloxy)-6-chloropyridine (7.7 g, 94% yield). ¹H NMR (CD₃OD, 400 MHz): δ 8.09 (bs, 1H), 7.30 (bs, 1H), 4.91 (bs, 1H), 3.72-3.51 (m, 4H), 2.32-2.15 (m, 2H), 1.48 (s, 9H).

The (R)—N-tert-butoxycarbonyl-3-bromo-5-(pyrrolidin-3-yloxy)-6-chloropyridine was then dissolved in dichloromethane (40 mL) and treated with trifluoroacetic acid (20 mL) for 2 h at ambient temperature. The solvent was evaporated, and the residue was dissolved in methanol (40 mL). Amberlyst A26 (OH) (15 g) was added, and the mixture was stirred for 10 min. This was filtered, and filtrate was concentrated to obtain (R)-3-bromo-5-(pyrrolidin-3-yloxy)-6-chloropyridine (6.3 g).

### Example 5

### (R)-3-bromo-5-(1-(4,4,4-trifluorobutanoyl)pyrrolidin-3-yloxy)-6-chloro-pyridine

To a solution of (R)-3-bromo-5-(pyrrolidin-3-yloxy)-6-chloropyridine (6.30 g, 22.7 mmol), 4,4,4-trifluorobutyric acid (3.48 g, 24.5 mmol) and triethylamine (8.53 mL, 61.2 mmol) in dimethylformamide (40 mL) was added O-Benzotriazole-N,N,N′,N′-tetramethyluronium hexafluorophosphate (HBTU) (9.28 g, 24.5 mmol), and the mixture was stirred at ambient temperature for 16 h. The mixture was diluted with ethyl acetate (200 mL), washed with 10% aqueous sodium bicarbonate solution (100 mL), and concentrated. The residue was purified on a silica gel column, using ethyl acetate in hexanes, to obtain (R)-3-bromo-5-(1-(4,4,4-trifluorobutanoyl)pyrrolidin-3-yloxy)-6-chloro-pyridine (5.5 g, 60% yield) as off-white solid. ¹H NMR (CDCl₃, 400 MHz): δ 8.14-8.11 (m, 1H), 7.34-7.31 (m, 1H), 5.04-4.97 (m, 1H), 3.91-3.65 (m, 4H), 2.61-2.10 (m, 6H); LCMS (m/z): 402 (M+1), 404 (M+2).

### Example 6

### (2S,4E)-N-methyl-5-(5-((3R)-1-(4,4,4-trifluorobutanoyl)pyrrolidin-3-yloxy)-6-chloro-3-pyridinyl)-4-penten-2-amine

A mixture of (S)—N-tert-butoxycarbonyl-N-methyl-4-penten-2-amine (3.28 g, 16.4 mmol), (R)-3-bromo-5-(1-(4,4,4-trifluorobutanoyl)pyrrolidin-3-yloxy)-6-chloro-pyridine (5.50 g, 13.7 mmol), palladium acetate (0.310 g, 1.37 mmol), tricyclohexylphosphine (0.384 g, 1.37 mmol) and diisopropylethylamine (4.78 mL, 27.4 mmol) in N-methylpyrrolidinone (80 mL) was stirred at 130° C. for 8 h. The solution was filtered, and the filtrate was partitioned between ethyl acetate (150 mL) and water (300 mL). The organic layer was separated, and the aqueous layer was extracted with ethyl acetate (100 mL). The combined organic extracts were dried over anhydrous sodium sulfate and concentrated. The crude product was dissolved in dichloromethane (100 mL) and treated with trifluoroacetic acid (50 mL) for 2 h. The volatiles were evaporated, and the residue was purified by HPLC, using mixtures of 0.05% TFA in water and 0.05% TFA in 30/70 isopropanol/methanol to obtain (2S,4E)-N-methyl-5-(5-((3R)-1-(4,4,4-trifluorobutanoyl)pyrrolidin-3-yloxy)-6-chloro-3-pyridinyl)-4-penten-2-amine trifluoroacetate (2.7 g, 47% yield) as gum. ¹H NMR (CD₃OD, 400 MHz): δ 8.07-8.0 (m, 1H), 7.64-7.61 (m, 1H), 6.63 (d, J=16 Hz, 1H), 6.47-6.39 (m, 1H), 5.29-5.21 (m, 1H), 3.90-3.51 (m, 4H), 3.43-3.35 (m, 1H), 2.75 (s, 3H), 2.72-2.49 (m, 6H), 2.38-2.32 (m, 1H), 2.23-2.18 (m, 1H), 1.34 (d, J=6.7 Hz, 3H); LCMS (m/z): 420 (M+1), 422 (M+2).

### V. Biological Assays

### Example 7

### Characterization of Interactions at Nicotinic Acetylcholine Receptors

### Cell Lines

SH-EP1/human α4β2 (Eaton et al., 2003), SH-EP1/human α4β4 (Gentry et al., 2003), SH-EP1/α6β3β4α5 (Grinevich et al., 2005), TE671/RD and SH-SY5Y cell lines (obtained from Dr. Ron Lukas, Barrow Neurological Institute) were maintained in proliferative growth phase in Dulbecco's modified Eagle's medium (Gibco/BRL) with 10% horse serum (Gibco BRL), 5% fetal bovine serum (HyClone, Logan Utah), 1 mM sodium pyruvate, 4 mM L-glutamine. For maintenance of stable transfectants, the α4β2 and α4β4 cell media was supplemented with 0.25 mg/mL zeocin and 0.13 mg/mL hygromycin B. Selection was maintained for the α6β3β4α5 cells with 0.25 mg/mL of zeocin, 0.13 mg/mL of hygromycin B, 0.4 mg/mL of geneticin, and 0.2 mg/mL of blasticidin. HEK/human α7/RIC3 cells (obtained from J. Lindstrom, U. Pennsylvania) were maintained in proliferative growth phase in Dulbecco's modified Eagle's medium (Gibco/BRL) with 10% fetal bovine serum (HyClone, Logan Utah), 1 mM sodium pyruvate, 4 mM L-glutamine, 0.4 mg/mL geneticin; 0.2 mg/ml hygromycin B.

### Receptor Binding Assays

Preparation of Membranes from Rat Tissues.

Rat cortices were obtained from Analytical Biological Services, Incorporated (ABS, Wilmington, Del.). Tissues were dissected from female Sprague-Dawley rats, frozen and shipped on dry ice. Tissues were stored at −20° C. until needed for membrane preparation. Cortices from 10 rats were pooled and homogenized by Polytron (Kinematica GmbH, Switzerland) in 10 volumes (weight:volume) of ice-cold preparative buffer (KCl, 11 mM; KH₂PO₄, 6 mM; NaCl 137 mM; Na₂HPO₄ 8 mM; HEPES (free acid), 20 mM; iodoacetamide, 5 mM; EDTA, 1.5 mM; 0.1 mM PMSF pH 7.4). The resulting homogenate was centrifuged at 40,000 g for 20 minutes at 4° C. and the resulting pellet was resuspended in 20 volumes of ice-cold water. After 60-minute incubation at 4° C., a new pellet was collected by centrifugation at 40,000 g for 20 minutes at 4° C. The final pellet was resuspended in preparative buffer and stored at −20° C. On the day of the assay, tissue was thawed, centrifuged at 40,000 g for 20 minutes and then resuspended in PBS (Dulbecco's Phosphate Buffered Saline, Life Technologies, pH 7.4) to a final concentration of 2-3 mg protein/mL. Protein concentrations were determined using the Pierce BCA Protein Assay kit (Pierce Biotechnology, Rockford, Ill.), with bovine serum albumin as the standard.

Preparation of Membranes from Clonal Cell Lines.

Cells were harvested in ice-cold PBS, pH 7.4, then homogenized with a polytron (Brinkmann Instruments, Westbury, N.Y.). Homongenates were centrifuged at 40,000 g for 20 minutes (4° C.). The pellet was resuspended in PBS and protein concentration determined using the Pierce BCA Protein Assay kit (Pierce Biotechnology, Rockford, Ill.).

Competition Binding to Receptors in Membrane Preparations.

Binding to nicotinic receptors was assayed on membranes using standard methods adapted from published procedures (Lippiello and Fernandes, 1986; Davies et al., 1999). In brief, membranes were reconstituted from frozen stocks (approximately 0.2 mg protein) and incubated for 2 h on ice in 150 ml assay buffer (PBS) in the presence of competitor compound (0.001 nM to 100 mM) and radioligand. [³H]-nicotine (L-(−)-[N-methyl-3H]-nicotine, 69.5 Ci/mmol, Perkin-Elmer Life Sciences) was used for human α4β2 binding studies. [³H]-epibatidine (52 Ci/mmol, Perkin-Elmer Life Sciences) was used for binding studies at the other receptor subtypes. Incubation was terminated by rapid filtration on a multimanifold tissue harvester (Brandel, Gaithersburg, Md.) using GF/B filters presoaked in 0.33% polyethyleneimine (w/v) to reduce non-specific binding. Filters were washed 3 times and the radioactivity retained was determined by liquid scintillation counting.

Binding Data Analysis.

Binding data were expressed as percent total control binding. Replicates for each point were averaged and plotted against the log of drug concentration. The IC₅₀ (concentration of the compound that produces 50% inhibition of binding) was determined by least squares non-linear regression using GraphPad Prism software (GraphPAD, San Diego, Calif.). Kᵢ was calculated using the Cheng-Prusoff equation (Cheng and Prusoff, 1973).

### Example 8

### Tabular Receptor Binding Data

The above illustrated synthetic procedures and other similar procedures were used to make the compounds shown in Table 1. Reagents and conditions will be readily apparent to those skilled in the art. In some cases, compounds were characterized by nuclear magnetic resonance (NMR) data. In other cases, compounds were structurally characterized by LCMS.

TABLE 1

| | Human | Human | Human | Rat |
|---|---|---|---|---|
| | α4β2 | α7 nAChR | Ganglion | Ganglion |
| Structure | Kᵢ (nM) | Kᵢ (nM) | Kᵢ (nM) | Kᵢ (nM) |
| 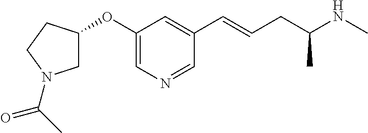 [SMILES: CN[C@@H](C)C/C=C/c1cncc(O[C@H]2CCN(C(C)=O)C2)c1] | 12 | — | — | — |
| 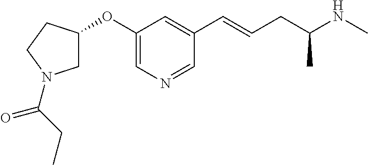 [SMILES: CCC(=O)N1CC[C@H](Oc2cncc(/C=C/C[C@H](C)NC)c2)C1] | 9.7 | — | 43000 | 40000 |
| 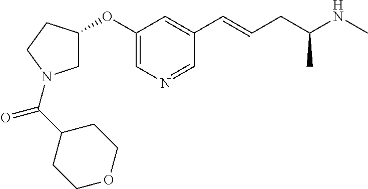 [SMILES: CN[C@@H](C)C/C=C/c1cncc(O[C@H]2CCN(C(=O)C3CCOCC3)C2)c1] | 13 | — | 100000 | 51000 |
| 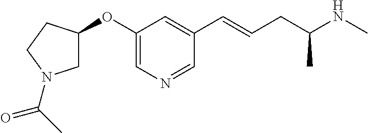 [SMILES: CN[C@@H](C)C/C=C/c1cncc(O[C@@H]2CCN(C(C)=O)C2)c1] | 2.6 | — | 41000 | 24000 |
| 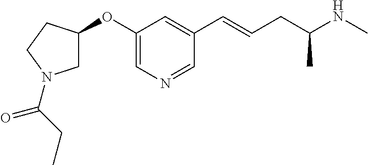 [SMILES: CCC(=O)N1CC[C@@H](Oc2cncc(/C=C/C[C@H](C)NC)c2)C1] | 1.9 | — | 19000 | 88000 |
| 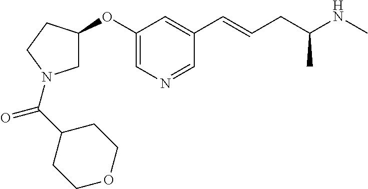 [SMILES: CN[C@@H](C)C/C=C/c1cncc(O[C@@H]2CCN(C(=O)C3CCOCC3)C2)c1] | 4.4 | — | 44000 | — |
| 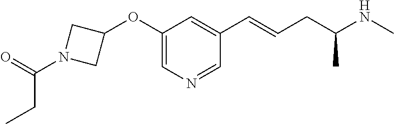 [SMILES: CCC(=O)N1CC(Oc2cncc(/C=C/C[C@H](C)NC)c2)C1] | 2.2 | — | — | — |
| 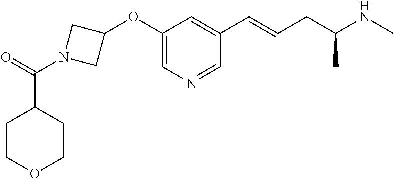 [SMILES: CN[C@@H](C)C/C=C/c1cncc(OC2CN(C(=O)C3CCOCC3)C2)c1] | 4.8 | — | — | — |
| 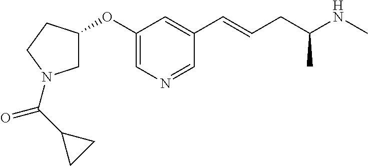 [SMILES: CN[C@@H](C)C/C=C/c1cncc(O[C@H]2CCN(C(=O)C3CC3)C2)c1] | 14 | — | 20000 | — |
| 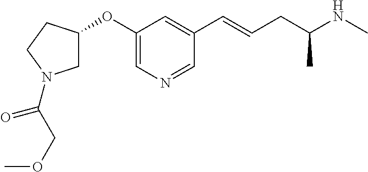 | 4.4 | — | 21000 | — |
| 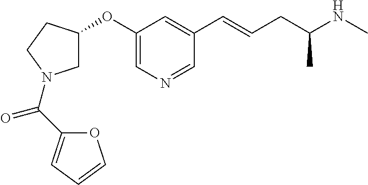 [SMILES: CN[C@@H](C)C/C=C/c1cncc(O[C@H]2CCN(C(=O)c3ccco3)C2)c1] | 1.2 | — | 72000 | — |
| 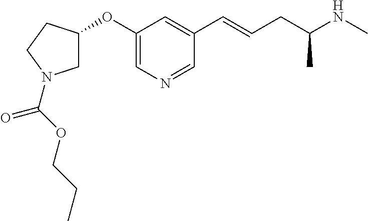 [SMILES: CCCOC(=O)N1CC[C@H](Oc2cncc(/C=C/C[C@H](C)NC)c2)C1] | 7.7 | — | 25000 | — |
| 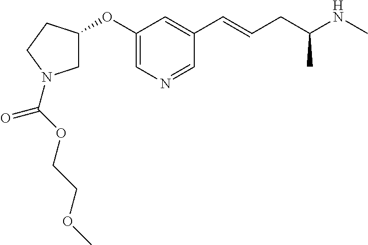 [SMILES: CN[C@@H](C)C/C=C/c1cncc(O[C@H]2CCN(C(=O)OCCOC)C2)c1] | 53 | — | 270000 | — |
| 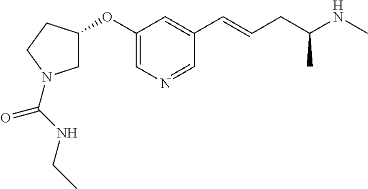 [SMILES: CCNC(=O)N1CC[C@H](Oc2cncc(/C=C/C[C@H](C)NC)c2)C1] | 61 | — | 250000 | — |
| 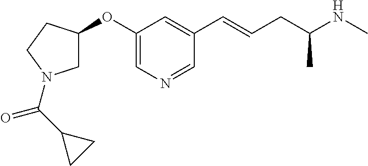 [SMILES: CN[C@@H](C)C/C=C/c1cncc(O[C@@H]2CCN(C(=O)C3CC3)C2)c1] | 5.1 | — | 83000 | — |
| 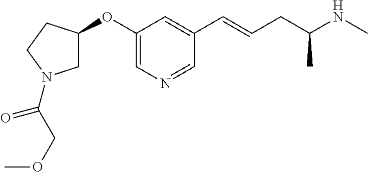 | 9.1 | — | 55000 | — |
| 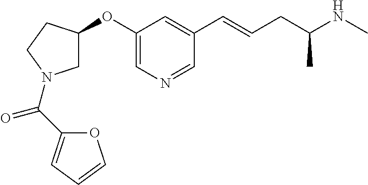 [SMILES: CN[C@@H](C)C/C=C/c1cncc(O[C@@H]2CCN(C(=O)c3ccco3)C2)c1] | 4.7 | — | 50000 | — |
| 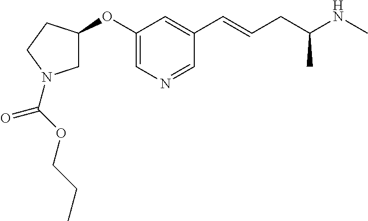 [SMILES: CCCOC(=O)N1CC[C@@H](Oc2cncc(/C=C/C[C@H](C)NC)c2)C1] | 5.1 | — | 37000 | — |
| 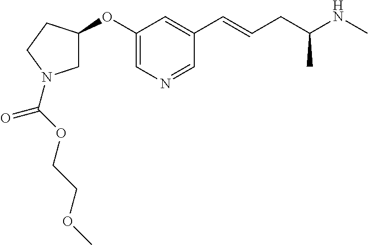 [SMILES: CN[C@@H](C)C/C=C/c1cncc(O[C@@H]2CCN(C(=O)OCCOC)C2)c1] | 6.6 | — | 48000 | — |
| 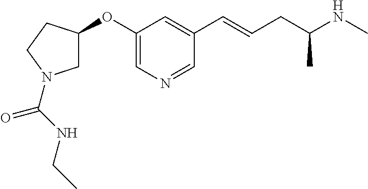 [SMILES: CCNC(=O)N1CC[C@@H](Oc2cncc(/C=C/C[C@H](C)NC)c2)C1] | 9.9 | — | 140000 | — |
| 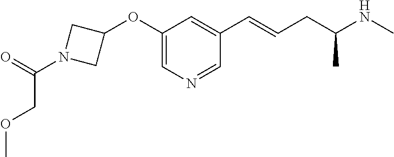 [SMILES: CN[C@@H](C)C/C=C/c1cncc(OC2CN(C(=O)COC)C2)c1] | 4.9 | — | 42000 | — |
| 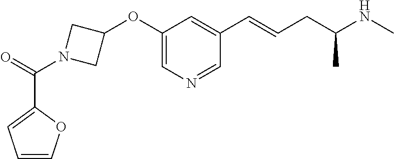 [SMILES: CN[C@@H](C)C/C=C/c1cncc(OC2CN(C(=O)c3ccco3)C2)c1] | 2.8 | — | 20000 | — |
| 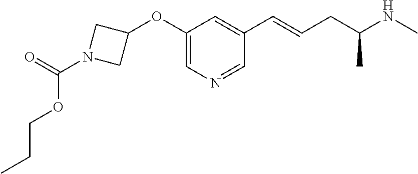 [SMILES: CCCOC(=O)N1CC(Oc2cncc(/C=C/C[C@H](C)NC)c2)C1] | 3.9 | — | 44000 | — |
| 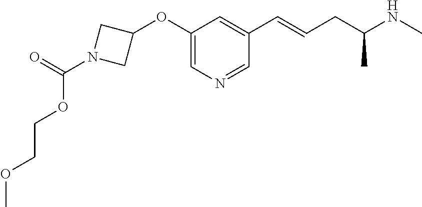 [SMILES: CN[C@@H](C)C/C=C/c1cncc(OC2CN(C(=O)OCCOC)C2)c1] | 9.6 | — | 69000 | — |
| 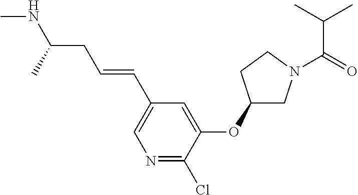 [SMILES: CN[C@@H](C)C/C=C/c1cnc(Cl)c(O[C@H]2CCN(C(=O)C(C)C)C2)c1] | 13 | 29000 | 27000 | 16000 |
| 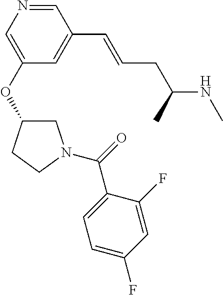 [SMILES: CN[C@@H](C)C/C=C/c1cncc(O[C@H]2CCN(C(=O)c3ccc(F)cc3F)C2)c1] | 29 | 47000 | 3300 | 80000 |
| 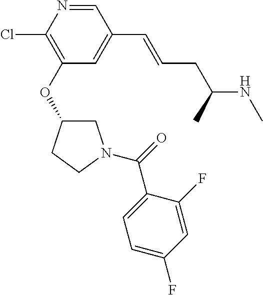 [SMILES: CN[C@@H](C)C/C=C/c1cnc(Cl)c(O[C@H]2CCN(C(=O)c3ccc(F)cc3F)C2)c1] | 22 | 23000 | — | — |
| 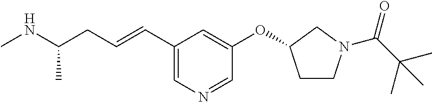 [SMILES: CN[C@@H](C)C/C=C/c1cncc(O[C@H]2CCN(C(=O)C(C)(C)C)C2)c1] | 7.5 | 100000 | 19000 | 130000 |
| 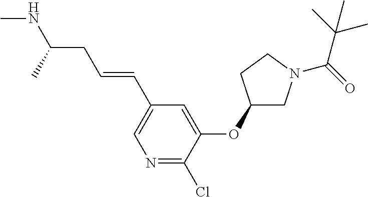 [SMILES: CN[C@@H](C)C/C=C/c1cnc(Cl)c(O[C@H]2CCN(C(=O)C(C)(C)C)C2)c1] | 6.7 | 39000 | 4800 | 11000 |
| 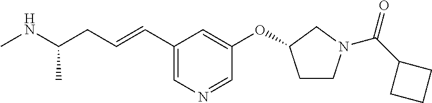 [SMILES: CN[C@@H](C)C/C=C/c1cncc(O[C@H]2CCN(C(=O)C3CCC3)C2)c1] | 18 | 66000 | 27000 | 17000 |
| 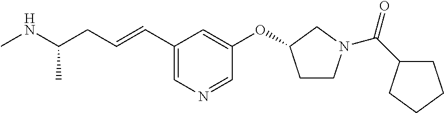 [SMILES: CN[C@@H](C)C/C=C/c1cncc(O[C@H]2CCN(C(=O)C3CCCC3)C2)c1] | 7.8 | 85000 | 13000 | 6900 |
| 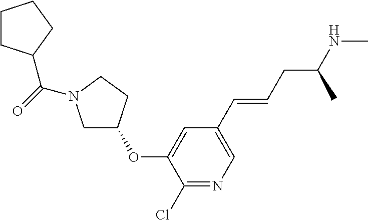 [SMILES: CN[C@@H](C)C/C=C/c1cnc(Cl)c(O[C@H]2CCN(C(=O)C3CCCC3)C2)c1] | 2.1 | 160000 | 7600 | 55000 |
| 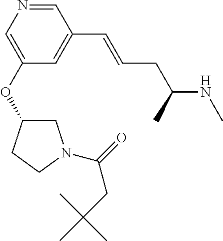 [SMILES: CN[C@@H](C)C/C=C/c1cncc(O[C@H]2CCN(C(=O)CC(C)(C)C)C2)c1] | 11 | 65000 | 12000 | 21000 |
| 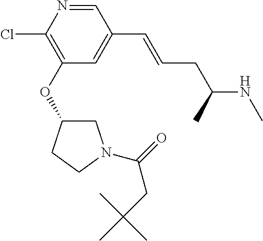 [SMILES: CN[C@@H](C)C/C=C/c1cnc(Cl)c(O[C@H]2CCN(C(=O)CC(C)(C)C)C2)c1] | 6.4 | 48000 | 15000 | 11000 |
| 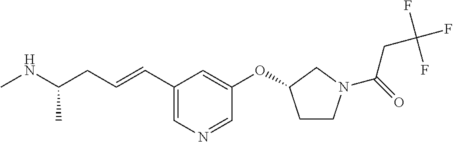 [SMILES: CN[C@@H](C)C/C=C/c1cncc(O[C@H]2CCN(C(=O)CC(F)(F)F)C2)c1] | 3.0 | 68000 | 5400 | 6800 |
| 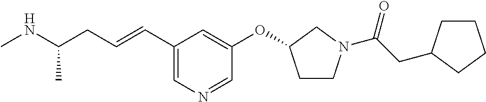 [SMILES: CN[C@@H](C)C/C=C/c1cncc(O[C@H]2CCN(C(=O)CC3CCCC3)C2)c1] | 8.6 | 49000 | 26000 | 11000 |
| 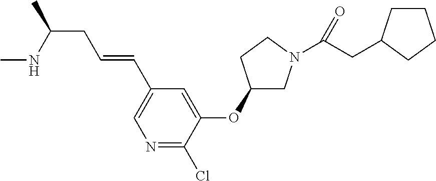 [SMILES: CN[C@@H](C)C/C=C/c1cnc(Cl)c(O[C@H]2CCN(C(=O)CC3CCCC3)C2)c1] | 6.1 | 36000 | 2500 | 4000 |
| 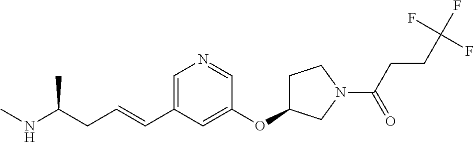 [SMILES: CN[C@@H](C)C/C=C/c1cncc(O[C@H]2CCN(C(=O)CCC(F)(F)F)C2)c1] | 11 | 49000 | 18000 | 33000 |
| 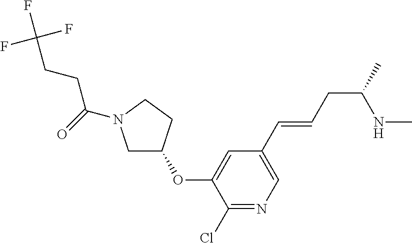 [SMILES: CN[C@@H](C)C/C=C/c1cnc(Cl)c(O[C@H]2CCN(C(=O)CCC(F)(F)F)C2)c1] | 2.8 | 13000 | 25000 | 31000 |
| 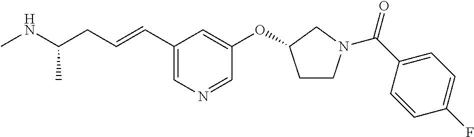 [SMILES: CN[C@@H](C)C/C=C/c1cncc(O[C@H]2CCN(C(=O)c3ccc(F)cc3)C2)c1] | 5.2 | 58000 | 14000 | 190000 |
| 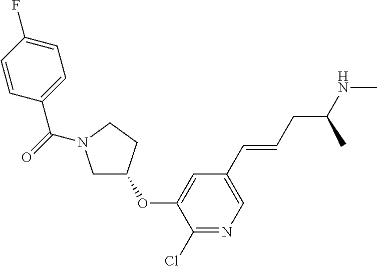 [SMILES: CN[C@@H](C)C/C=C/c1cnc(Cl)c(O[C@H]2CCN(C(=O)c3ccc(F)cc3)C2)c1] | 1.5 | 98000 | 16000 | 5900 |
| 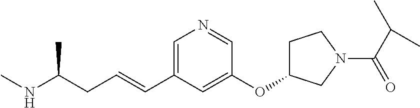 [SMILES: CN[C@@H](C)C/C=C/c1cncc(O[C@@H]2CCN(C(=O)C(C)C)C2)c1] | 9.0 | 35000 | 11000 | 50000 |
| 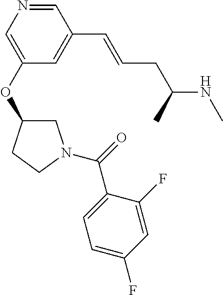 [SMILES: CN[C@@H](C)C/C=C/c1cncc(O[C@@H]2CCN(C(=O)c3ccc(F)cc3F)C2)c1] | 5.9 | 41000 | 3000 | 19000 |
| 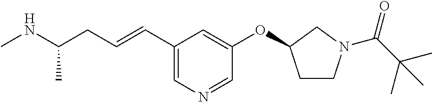 [SMILES: CN[C@@H](C)C/C=C/c1cncc(O[C@@H]2CCN(C(=O)C(C)(C)C)C2)c1] | 17 | 51000 | 21000 | 100000 |
| 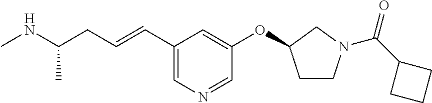 [SMILES: CN[C@@H](C)C/C=C/c1cncc(O[C@@H]2CCN(C(=O)C3CCC3)C2)c1] | 6.1 | 58000 | 26000 | 45000 |
| 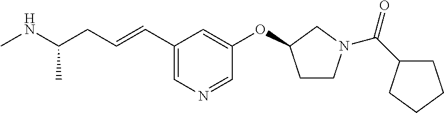 [SMILES: CN[C@@H](C)C/C=C/c1cncc(O[C@@H]2CCN(C(=O)C3CCCC3)C2)c1] | 7.4 | 26000 | 14000 | 28000 |
| 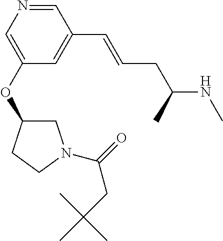 [SMILES: CN[C@@H](C)C/C=C/c1cncc(O[C@@H]2CCN(C(=O)CC(C)(C)C)C2)c1] | 32 | 260000 | 43000 | 35000 |
| 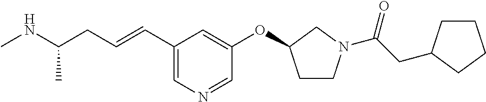 [SMILES: CN[C@@H](C)C/C=C/c1cncc(O[C@@H]2CCN(C(=O)CC3CCCC3)C2)c1] | 11 | 53000 | 5800 | 170000 |
| 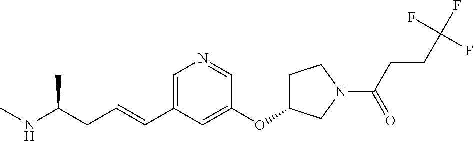 [SMILES: CN[C@@H](C)C/C=C/c1cncc(O[C@@H]2CCN(C(=O)CCC(F)(F)F)C2)c1] | 4.3 | 170000 | 19000 | 43000 |
| 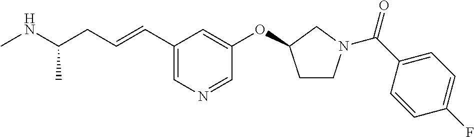 [SMILES: CN[C@@H](C)C/C=C/c1cncc(O[C@@H]2CCN(C(=O)c3ccc(F)cc3)C2)c1] | 6.7 | 53000 | 6800 | 100000 |
| 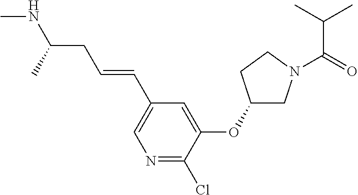 [SMILES: CN[C@@H](C)C/C=C/c1cnc(Cl)c(O[C@@H]2CCN(C(=O)C(C)C)C2)c1] | 1.5 | 36000 | — | — |
| 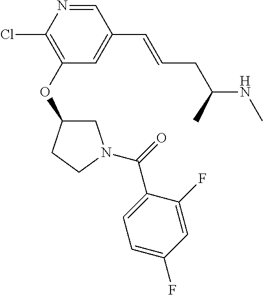 [SMILES: CN[C@@H](C)C/C=C/c1cnc(Cl)c(O[C@@H]2CCN(C(=O)c3ccc(F)cc3F)C2)c1] | 3.8 | 22000 | 6600 | 22000 |
| 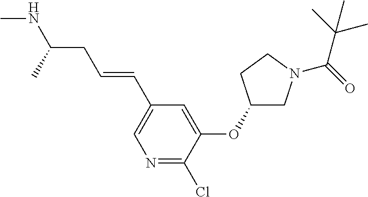 [SMILES: CN[C@@H](C)C/C=C/c1cnc(Cl)c(O[C@@H]2CCN(C(=O)C(C)(C)C)C2)c1] | 8.7 | 34000 | 26000 | 1100 |
| 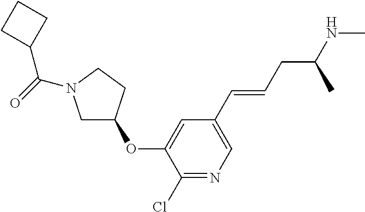 [SMILES: CN[C@@H](C)C/C=C/c1cnc(Cl)c(O[C@@H]2CCN(C(=O)C3CCC3)C2)c1] | 1.5 | 45000 | — | — |
| 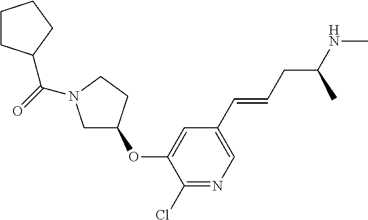 [SMILES: CN[C@@H](C)C/C=C/c1cnc(Cl)c(O[C@@H]2CCN(C(=O)C3CCCC3)C2)c1] | 2.9 | 6100 | — | — |
| 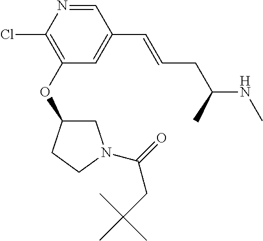 [SMILES: CN[C@@H](C)C/C=C/c1cnc(Cl)c(O[C@@H]2CCN(C(=O)CC(C)(C)C)C2)c1] | 16 | 6500 | — | — |
| 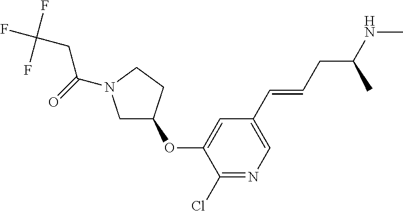 [SMILES: CN[C@@H](C)C/C=C/c1cnc(Cl)c(O[C@@H]2CCN(C(=O)CC(F)(F)F)C2)c1] | 1.2 | 300000 | 18000 | 8500 |
| 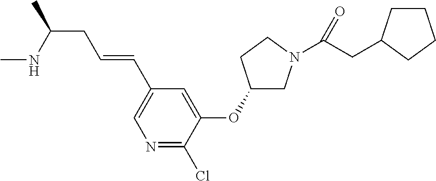 [SMILES: CN[C@@H](C)C/C=C/c1cnc(Cl)c(O[C@@H]2CCN(C(=O)CC3CCCC3)C2)c1] | 7.5 | 30000 | — | — |
| 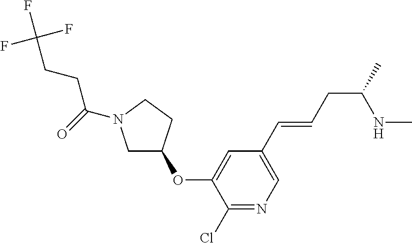 [SMILES: CN[C@@H](C)C/C=C/c1cnc(Cl)c(O[C@@H]2CCN(C(=O)CCC(F)(F)F)C2)c1] | 1.4 | 55000 | 7400 | 15000 |
| 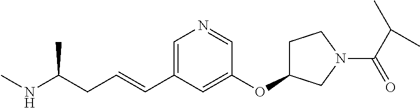 [SMILES: CN[C@@H](C)C/C=C/c1cncc(O[C@H]2CCN(C(=O)C(C)C)C2)c1] | 6.1 | 36000 | 20000 | 5700 |
| 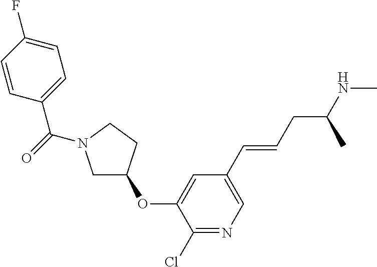 [SMILES: CN[C@@H](C)C/C=C/c1cnc(Cl)c(O[C@@H]2CCN(C(=O)c3ccc(F)cc3)C2)c1] | 3.3 | 61000 | 2000 | 3700 |

### Summary of Binding Data

Compounds of Table 1, representative of the present invention, exhibited inhibition constants (Ki values) at the human α4β2 subtype in the ranges of 1.2 nM to 61 nM, indicating high affinity for the α4β2 subtype. Ki values at the α7 subtype were >13,000 nM, indicating low affinity for the α7 subtype. Likewise, the affinity of these compounds for the ganglionic subtype (both rat and human) was typically low (Ki values >1000 nM) and often very low (Ki values >10,000 nM).

### Example 9

### Morris Water Maze

Compounds of the present invention were tested in the Morris Water Navigation Task to characterize the cognitive-enhancing properties, namely through a chemically-induced memory (spatial) deficit task in the mouse. Reference may be had to Morris, R. G. M. (1981), Spatial Localization Does Not Require the Presence of Local Cues, Learning and Motivation, 12(2), 239-260; and R. G. M. Morris, P. Garrud, J. N. P. Rawlins & J. O'Keefe (1982), Place Navigation Impaired in Rats with Hippocampal Lesions, Nature, 297, 681-683.

The animals were male CD-1 mice (Charles River, Raleigh, N.C., USA), with 25-30 g body weight range on receipt. Animals were stabilized for at least 5 days after delivery in plastic cages on alpha-dry bedding with free access to food and water.

Test Compound, (2S,4E)-N-methyl-5-(5-((3R)-1-(4,4,4-trifluorobutanoyl)pyrrolidin-3-yloxy)-6-chloro-3-pyridinyl)-4-penten-2-amine, or a pharmaceutically acceptable salt thereof, such as the hemi-galactarate, was dissolved in deionized water (salt/base ratio=1.25).

The treatment schedule included scopolamine (0.75 mg/kg; s.c.) administered 15 minutes before the first training session each training day in combination with Test Compound (0.3, 1, 3 mg/kg of base) administered i.g. 25 minutes before the first training session each training day.

Scopolamine (0.75 mg/kg; s.c.) alone was administered 15 minutes before the training session each training day.

Cognitive activity was evaluated using an acquisition procedure in the Morris water maze whereby animals are administered a cognitively impairing compound (scopolamine) alone or in combination with a Test Compound during training sessions on days 1-4. Performance is evaluated on day 5 during a probe trial without any drug on board.

Preliminary analysis used one-way analysis of variance followed by post-hoc t-tests (Dunnetts) where applicable.

### Results:

There was a significant overall drug effect in both proximity to the platform location (F(3, 43)=6.44, p<0.01) and % time spent in the target quadrant (F(3,43)=4.80, p<0.01) during the probe trial.

Animals treated with Test Compound (0.3 mg/kg; i.g.) in combination with scopolamine (0.75 mg/kg; s.c.) showed significantly improved performance in this water maze task as indicated by smaller mean distance from the platform (proximity) in the probe trial compared with animals treated with scopolamine alone (Dunnett's p<0.01).

Animals treated with Test Compound (0.3 and 3.0 mg/kg; i.g.) in combination with scopolamine (0.75 mg/kg; s.c.) showed significantly improved performance in this water maze task as indicated by an increased time spent in the target quadrant in the probe trial compared with animals treated with scopolamine alone (Dunnett's p<0.01 and p<0.05 respectively).

One animal was found dead on the morning of Training Trial 2.

Assessment of cognitive effects in a spatial memory paradigm demonstrated that Test Compound, (2S,4E)-N-methyl-5-(5-((3R)-1-(4,4,4-trifluorobutanoyl)pyrrolidin-3-yloxy)-6-chloro-3-pyridinyl)-4-penten-2-amine hemi-galactarate, at doses of 0.3 and 3.0 mg/kg significantly improved spatial memory performance in rats cognitively impaired by scopolamine.

### Example 10

### Objection Recognition Task

The compounds of the present invention were assessed for cognitive enhancing properties in the object recognition task in rats. In more detail, the compounds of the present invention, specifically Test Compound, (2S,4E)-N-methyl-5-(5-((3R)-1-(4,4,4-trifluorobutanoyl)pyrrolidin-3-yloxy)-6-chloro-3-pyridinyl)-4-penten-2-amine, or a pharmaceutically acceptable salt thereof, such as the hemi-galactarate, were tested to characterize the cognitive-enhancing properties of in a memory (recognition) task in the rat following oral dosing. Reference is made, for example, to Ennaceur A., et al., (1988), A New One-Trial Test for Neurobiological Studies of Memory in Rats, Behavioral Brain Research, 31, pp. 47-59.

The animals used were male Sprague-Dawley rats (Charles River, Raleigh, N.C., USA), of 134-173 g body weight range on receipt. The animals were stabilized for at least 6 days after delivery in plastic cages on alpha-dry bedding with access to food and water (2 per cage).

The Test Compound was dissolved in deionized water. The treatment schedule was 0.001, 0.01 and 0.1 mg/kg of base (salt/base ratio=1.25; equivalent to 0.0023, 0.023, 0.23 mmol/kg) or vehicle was administered p.o. 6H before exploratory, acquisition, and recall object recognition trials. All treatments were separated by 24 h, however although the acclimation and acquisition trials were separated by 24 h, the recall trial occurred 30 h after the acquisition trial (i.e. 6 h after the third dose).

The method of evaluation included cognitive activity using the method of Luine et al. A statistical analysis was performed using parametric distribution (exploration time): Student's t-test; (% RI): Kruskall-Wallace One-way ANOVA on Ranks with Dunn's method for post-hoc analyses.

The results included an average time spent on object A versus object B by vehicle-treated group after the acquisition session was not significantly different (P=0.76). By contrast, animals treated with any dose of Test Compound (0.001, 0.01, or 0.1 1 mg/kg) spent significantly (P<0.05) more time investigating object B (novel) than object A (familiar). Recognition index was improved in all groups treated with Test Compound compared to the recognition index (51%) of the vehicle-treated group, with statistical significance at the two higher treatment doses. No overt behavioral side effects were observed. Data from animals having a total exploration time (object A+object B) of less than 5 sec were not included in the analyses.

Assessment of cognitive effects in an object recognition paradigm demonstrated that Test Compound facilitates episodic memory in young rats in the dose range of 0.01 to 0.1 mg/kg for % RI, with a strong trend at the lower dose of 0.001 mg/kg. Based on non-parametric statistical analyses for % RI, the MED was 0.01 mg/kg, however, the lower dose of 0.001 was very nearly significant, suggesting an MED somewhere between 0.001 and 0.01 mg/kg in this paradigm.

The specific pharmacological responses observed may vary according to and depending on the particular active compound selected or whether there are present pharmaceutical carriers, as well as the type of formulation and mode of administration employed, and such expected variations or differences in the results are contemplated in accordance with practice of the present invention.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A compound of Formula I: 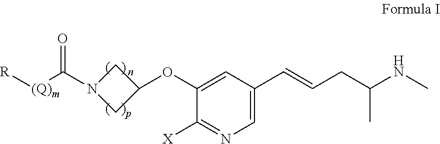
wherein:
n is 0, 1, 2, 3, or 4;
p is 0, 1, 2, 3, or 4;
and the sum of n and p is 2, 3, or 4;
m is 0 or 1;
Q is —CH₂—, —NH— or —O—;
X is H or halogen;
R is chosen from the group consisting of
C₁₋₆ alkyl, optionally substituted with one or more fluorine atoms,
C₃₋₆ cycloalkyl, optionally substituted with one or more fluorine atoms,
C₃₋₆ heterocyclyl, optionally substituted with one or more of fluorine, C₁₋₆ alkyloxy, and C₁₋₆ alkyl optionally substituted with one or more fluorine atoms,
heteroaryl, optionally substituted with one or more fluorine atoms, and
aryl, optionally substituted with one or more fluorine atoms;
or a pharmaceutically acceptable salt thereof.

2. The compound of claim 1 where m is 0.

3. The compound of claim 1 where m is 1 and Q is —O—.

4. A compound selected from:
(2S,4E)-N-methyl-5-(5-((3R)-1-acetylpyrrolidin-3-yloxy)-3-pyridinyl)-4-penten-2-amine;
(2S,4E)-N-methyl-5-(5-((3R)-1-propanoylpyrrolidin-3-yloxy)-3-pyridinyl)-4-penten-2-amine;
(2S,4E)-N-methyl-5-(5-((3R)-1-(tetrahydro-2H-pyran-4-carbonyl)pyrrolidin-3-yloxy)-3-pyridinyl)-4-penten-2-amine;
(2S,4E)-N-methyl-5-(5-((3S)-1-acetylpyrrolidin-3-yloxy)-3-pyridinyl)-4-penten-2-amine;
(2S,4E)-N-methyl-5-(5-((3S)-1-propanoylpyrrolidin-3-yloxy)-3-pyridinyl)-4-penten-2-amine;
(2S,4E)-N-methyl-5-(5-((3S)-1-(tetrahydro-2H-pyran-4-carbonyl)pyrrolidin-3-yloxy)-3-pyridinyl)-4-penten-2-amine;
(2S,4E)-N-methyl-5-(5-(1-propanoylazetidin-3-yloxy)-3-pyridinyl)-4-penten-2-amine;
(2S,4E)-N-methyl-5-(5-(1-(tetrahydro-2H-pyran-4-carbonyl)azetidin-3-yloxy)-3-pyridinyl)-4-penten-2-amine;
(2S,4E)-N-methyl-5-(5-((3R)-1-(cyclopropylcarbonyl)pyrrolidin-3-yloxy)-3-pyridinyl)-4-penten-2-amine;
(2S,4E)-N-methyl-5-(5-((3R)-1-(methoxyacetyl)pyrrolidin-3-yloxy)-3-pyridinyl)-4-penten-2-amine;
(2S,4E)-N-methyl-5-(5-((3R)-1-(furan-2-ylcarbonyl)pyrrolidin-3-yloxy)-3-pyridinyl)-4-penten-2-amine;
(2S,4E)-N-methyl-5-(5-((3R)-1-(propoxycarbonyl)pyrrolidin-3-yloxy)-3-pyridinyl)-4-penten-2-amine;
(2S,4E)-N-methyl-5-(5-((3R)-1-(methoxyethoxycarbonyl)pyrrolidin-3-yloxy)-3-pyridinyl)-4-penten-2-amine;
(2S,4E)-N-methyl-5-(5-((3R)-1-(ethylaminocarbonyl)pyrrolidin-3-yloxy)-3-pyridinyl)-4-penten-2-amine;
(2S,4E)-N-methyl-5-(5-((3S)-1-(cyclopropylcarbonyl)pyrrolidin-3-yloxy)-3-pyridinyl)-4-penten-2-amine;
(2S,4E)-N-methyl-5-(5-((3S)-1-(methoxyacetyl)pyrrolidin-3-yloxy)-3-pyridinyl)-4-penten-2-amine;
(2S,4E)-N-methyl-5-(5-((3S)-1-(furan-2-yl-carbonyl)pyrrolidin-3-yloxy)-3-pyridinyl)-4-penten-2-amine;
(2S,4E)-N-methyl-5-(5-((3S)-1-(propoxycarbonyl)pyrrolidin-3-yloxy)-3-pyridinyl)-4-penten-2-amine;
(2S,4E)-N-methyl-5-(5-((3S)-1-(methoxyethoxycarbonyl)pyrrolidin-3-yloxy)-3-pyridinyl)-4-penten-2-amine;
(2S,4E)-N-methyl-5-(5-((3S)-1-(ethylaminocarbonyl)pyrrolidin-3-yloxy)-3-pyridinyl)-4-penten-2-amine;
(2S,4E)-N-methyl-5-(5-(1-(methoxyacetyl)azetidin-3-yloxy)-3-pyridinyl)-4-penten-2-amine;
(2S,4E)-N-methyl-5-(5-(1-(furan-2-yl-carbonyl)azetidin-3-yloxy)-3-pyridinyl)-4-penten-2-amine;
(2S,4E)-N-methyl-5-(5-(1-(propoxycarbonyl)azetidin-3-yloxy)-3-pyridinyl)-4-penten-2-amine;
(2S,4E)-N-methyl-5-(5-(1-(methoxyethoxycarbonyl)azetidin-3-yloxy)-3-pyridinyl)-4-penten-2-amine;
(2S,4E)-N-methyl-5-(5-((3S)-1-(isopropylcarbonyl)pyrrolidin-3-yloxy)-6-chloro-3-pyridinyl)-4-penten-2-amine;
(2S,4E)-N-methyl-5-(5-((3S)-1-(2,4-difluorobenzoyl)pyrrolidin-3-yloxy)-3-pyridinyl)-4-penten-2-amine;
(2S,4E)-N-methyl-5-(5-((3S)-1-(2,4-difluorobenzoyl)pyrrolidin-3-yloxy)-6-chloro-3-pyridinyl)-4-penten-2-amine;
(2S,4E)-N-methyl-5-(5-((3S)-1-(tert-butylcarbonyl)pyrrolidin-3-yloxy)-3-pyridinyl)-4-penten-2-amine;
(2S,4E)-N-methyl-5-(5-((3S)-1-(tert-butylcarbonyl)pyrrolidin-3-yloxy)-6-chloro-3-pyridinyl)-4-penten-2-amine;
(2S,4E)-N-methyl-5-(5-((3S)-1-(cyclobutylcarbonyl)pyrrolidin-3-yloxy)-3-pyridinyl)-4-penten-2-amine;
(2S,4E)-N-methyl-5-(5-((3S)-1-(cyclopentylcarbonyl)pyrrolidin-3-yloxy)-3-pyridinyl)-4-penten-2-amine;
(2S,4E)-N-methyl-5-(5-((3S)-1-(cyclopentylcarbonyl)pyrrolidin-3-yloxy)-6-chloro-3-pyridinyl)-4-penten-2-amine;
(2S,4E)-N-methyl-5-(5-((3S)-1-(tert-butylacetyl)pyrrolidin-3-yloxy)-3-pyridinyl)-4-penten-2-amine;
(2S,4E)-N-methyl-5-(5-((3S)-1-(tert-butylacetyl)pyrrolidin-3-yloxy)-6-chloro-3-pyridinyl)-4-penten-2-amine;
(2S,4E)-N-methyl-5-(5-((3S)-1-((3,3,3-trifluoropropanoyl)pyrrolidin-3-yloxy)-3-pyridinyl)-4-penten-2-amine;
(2S,4E)-N-methyl-5-(5-((3S)-1-(cyclopentylacetyl)pyrrolidin-3-yloxy)-3-pyridinyl)-4-penten-2-amine;
(2S,4E)-N-methyl-5-(5-((3S)-1-(cyclopentylacetyl)pyrrolidin-3-yloxy)-6-chloro-3-pyridinyl)-4-penten-2-amine;
(2S,4E)-N-methyl-5-(5-((3S)-1-(4,4,4-trifluorobutanoyl)pyrrolidin-3-yloxy)-3-pyridinyl)-4-penten-2-amine;
(2S,4E)-N-methyl-5-(5-((3S)-1-(4-fluorobenzoyl)pyrrolidin-3-yloxy)-3-pyridinyl)-4-penten-2-amine;
(2S,4E)-N-methyl-5-(5-((3S)-1-(4-fluorobenzoyl)pyrrolidin-3-yloxy)-6-chloro-3-pyridinyl)-4-penten-2-amine;
(2S,4E)-N-methyl-5-(5-((3R)-1-(isopropylcarbonyl)pyrrolidin-3-yloxy)-3-pyridinyl)-4-penten-2-amine;
(2S,4E)-N-methyl-5-(5-((3R)-1-(2,4-difluorobenzoyl)pyrrolidin-3-yloxy)-3-pyridinyl)-4-penten-2-amine;
(2S,4E)-N-methyl-5-(5-((3R)-1-(tert-butylcarbonyl)pyrrolidin-3-yloxy)-3-pyridinyl)-4-penten-2-amine;
(2S,4E)-N-methyl-5-(5-((3R)-1-(cyclobutylcarbonyl)pyrrolidin-3-yloxy)-3-pyridinyl)-4-penten-2-amine;
(2S,4E)-N-methyl-5-(5-((3R)-1-(cyclopentylcarbonyl)pyrrolidin-3-yloxy)-3-pyridinyl)-4-penten-2-amine;
(2S,4E)-N-methyl-5-(5-((3R)-1-(tert-butylacetyl)pyrrolidin-3-yloxy)-3-pyridinyl)-4-penten-2-amine;
(2S,4E)-N-methyl-5-(5-((3R)-1-(cyclopentylacetyl)pyrrolidin-3-yloxy)-3-pyridinyl)-4-penten-2-amine;
(2S,4E)-N-methyl-5-(5-((3R)-1-((4,4,4-trifluorobutanoyl)pyrrolidin-3-yloxy)-3-pyridinyl)-4-penten-2-amine;
(2S,4E)-N-methyl-5-(5-((3R)-1-(4-fluorobenzoyl)pyrrolidin-3-yloxy)-3-pyridinyl)-4-penten-2-amine;
(2S,4E)-N-methyl-5-(5-((3R)-1-(isopropylcarbonyl)pyrrolidin-3-yloxy)-6-chloro-3-pyridinyl)-4-penten-2-amine;
(2S,4E)-N-methyl-5-(5-((3R)-1-((2,4-difluorobenzoyl)pyrrolidin-3-yloxy)-6-chloro-3-pyridinyl)-4-penten-2-amine;
(2S,4E)-N-methyl-5-(5-((3R)-1-(tert-butylcarbonyl)pyrrolidin-3-yloxy)-6-chloro-3-pyridinyl)-4-penten-2-amine;
(2S,4E)-N-methyl-5-(5-((3R)-1-(cyclobutylcarbonyl)pyrrolidin-3-yloxy)-6-chloro-3-pyridinyl)-4-penten-2-amine;
(2S,4E)-N-methyl-5-(5-((3R)-1-(cyclopentylcarbonyl)pyrrolidin-3-yloxy)-6-chloro-3-pyridinyl)-4-penten-2-amine;
(2S,4E)-N-methyl-5-(5-((3R)-1-(tert-butylacetyl)pyrrolidin-3-yloxy)-6-chloro-3-pyridinyl)-4-penten-2-amine;
(2S,4E)-N-methyl-5-(5-((3R)-1-(3,3,3-trifluoropropanoyl)pyrrolidin-3-yloxy)-6-chloro-3-pyridinyl)-4-penten-2-amine;
(2S,4E)-N-methyl-5-(5-((3R)-1-(cyclopentylacetyl)pyrrolidin-3-yloxy)-6-chloro-3-pyridinyl)-4-penten-2-amine;
(2S,4E)-N-methyl-5-(5-((3S)-1-(isopropylcarbonyl)pyrrolidin-3-yloxy)-3-pyridinyl)-4-penten-2-amine; and
(2S,4E)-N-methyl-5-(5-((3R)-1-(4-fluorobenzoyl)pyrrolidin-3-yloxy)-6-chloro-3-pyridinyl)-4-penten-2-amine;
or a pharmaceutically acceptable salt thereof.

5. A pharmaceutical composition comprising a compound as claimed in claim 1 and a pharmaceutically acceptable carrier.

6. A method for the treatment or prevention of a disease or condition mediated by a neuronal nicotinic receptor comprising the administration of a compound as claimed in any claim 1.

7. The method of claim 6, wherein the neuronal nicotinic receptor is of the α4β2 subtype.

8. The method of claim 6, wherein the disease or condition is a CNS disorder.

9. The method of claim 6, wherein the disease or condition is selected from the group consisting of age-associated memory impairment, mild cognitive impairment, age-related cognitive decline, pre-senile dementia, early onset Alzheimer's disease, senile dementia, dementia of the Alzheimer's type, Alzheimer's disease, cognitive impairment no dementia (CIND), Lewy body dementia, HIV-dementia, AIDS dementia complex, vascular dementia, Down syndrome, head trauma, traumatic brain injury (TBI), dementia pugilistica, Creutzfeld-Jacob Disease and prion diseases, stroke, ischemia, attention deficit disorder, attention deficit hyperactivity disorder, dyslexia, schizophrenia, schizophreniform disorder, schizoaffective disorder, cognitive dysfunction in schizophrenia, cognitive deficits in schizophrenia, Parkinsonism including Parkinson's disease, postencephalitic parkinsonism, parkinsonism-dementia of Gaum, frontotemporal dementia Parkinson's Type (FTDP), Pick's disease, Niemann-Pick's Disease, Huntington's Disease, Huntington's chorea, tardive dyskinesia, hyperkinesia, progressive supranuclear palsy, progressive supranuclear paresis, restless leg syndrome, Creutzfeld-Jakob disease, multiple sclerosis, amyotrophic lateral sclerosis (ALS), motor neuron diseases (MND), multiple system atrophy (MSA), corticobasal degeneration, Guillain-Barré Syndrome (GBS), chronic inflammatory demyelinating polyneuropathy (CIDP), epilepsy, autosomal dominant nocturnal frontal lobe epilepsy, mania, anxiety, depression, premenstrual dysphoria, panic disorders, bulimia, anorexia, narcolepsy, excessive daytime sleepiness, bipolar disorders, generalized anxiety disorder, obsessive compulsive disorder, rage outbursts, oppositional defiant disorder, Tourette's syndrome, autism, drug and alcohol addiction, tobacco addiction, acute pain, chronic pain, and one or more neuropathies.

13. A compound N-methyl-5-(5-(1-(4,4,4-trifluorobutanoyl)pyrrolidin-3-yloxy)-6-chloro-3-pyridinyl)-4-penten-2-amine or a pharmaceutically acceptable salt thereof.

14. A compound (2S,4E)-N-methyl-5-(5-((3R)-1-(4,4,4-trifluorobutanoyl)pyrrolidin-3-yloxy)-6-chloro-3-pyridinyl)-4-penten-2-amine or a pharmaceutically acceptable salt thereof.

15. A compound (2S,4E)-N-methyl-5-(5-((3S)-1-(4,4,4-trifluorobutanoyl)pyrrolidin-3-yloxy)-6-chloro-3-pyridinyl)-4-penten-2-amine or a pharmaceutically acceptable salt thereof.

16. A pharmaceutical composition comprising a compound as claimed in claim 13, and a pharmaceutically acceptable carrier.

17. A pharmaceutical composition comprising a compound as claimed in claim 14, and a pharmaceutically acceptable carrier.

18. A pharmaceutical composition comprising a compound as claimed in claim 15, and a pharmaceutically acceptable carrier.

19. A method for the treatment or prevention of a disease or condition mediated by a neuronal nicotinic receptor comprising the administration of a compound as claimed in claim 13.

20. A method for the treatment or prevention of a disease or condition mediated by a neuronal nicotinic receptor comprising the administration of a compound as claimed in claim 14.

21. A method for the treatment or prevention of a disease or condition mediated by a neuronal nicotinic receptor comprising the administration of a compound as claimed in claim 15.
